(19) United States
(12) Patent Application Publication
(10) Pub. No.: US 2026/0109733 A1
(43) Pub. Date: Apr. 23, 2026

(54) MULTI-SCALE INTERACTOME PROFILING OF MEMBRANE PROTEINS USING PHOTOCATALYTIC PROXIMITY LABELING

(71) Applicant: THE REGENTS OF THE UNIVERSITY OF CALIFORNIA, Oakland, CA (US)

(72) Inventors: James Wells, Oakland, CA (US); Zhi Lin, Oakland, CA (US)

(21) Appl. No.: 19/413,455

(22) Filed: Dec. 9, 2025

Related U.S. Application Data
(63) Continuation-in-part of application No. PCT/US2024/033253, filed on Jun. 10, 2024.
(60) Provisional application No. 63/472,087, filed on Jun. 9, 2023.

(51) Int. Cl. C07K 1/13; G01N 33/58

(52) U.S. Cl. CPC C07K 1/13; G01N 33/582

(57) ABSTRACT

Photocatalysts for activating multiple photoreactive probes and methods of proximity labeling proteins on a cell surface using same.

### CROSS-REFERENCE TO RELATED APPLICATION

This application is a continuation-in-part of International Patent Application No PCT/US24/33253, filed Jun. 10, 2024, which claims the benefit under 35 U.S.C. § 119(e) of U.S. Provisional Patent No. 63/472,087, filed Jun. 9, 2023, the entire disclosures of which are incorporated herein by reference.

### STATEMENT OF GOVERNMENT INTEREST

This invention was made with government support under R35 GM122451 and R01 CA248323, awarded by the National Institutes of Health. The government has certain rights in the invention.

### FIELD

The disclosure generally relates to labeling of proteins. More specifically, the disclosure relates to the labeling and identification of proteins proximal to target protein using a photocatalyst on a cell surface.

### BACKGROUND

Protein interactions play a pivotal role in cellular signaling, especially on the cell surface. These interactions span a wide range of length scales, posing a challenge to map a given protein's interactome with maximal coverage and resolution. Although recent advances in mass spectrometry-based interactomics have improved the ability to discover proteins proximal to the target of interest, these methods are limited to mapping interactions within a discrete radius. As a result, distal, yet functionally relevant targets, may be overlooked, and background enrichment due to bystander effects can be significant. Tools to examine interactomes across multiple radii, while illuminating both proximal and distal interactions, remains inaccessible.

Further, existing tools for mapping protein interactions often necessitate protein engineering or the use of expensive metal catalysts such as Ir, Ru, Os, which are difficult to make. Accordingly, there is a need in the art for a single, cost-efficient organic photocatalyst, that can provide information from multiple datasets of interactome profiling across different length scales.

### SUMMARY

The disclosure provides methods of labeling a protein with a photoreactive probe, the methods including admixing a protein, a photoreactive probe, and photocatalyst to form a mixture; and irradiating the mixture with light having a wavelength in a range of about 410 nm to about 570 nm; wherein the photocatalyst has a structure according to formula (I):

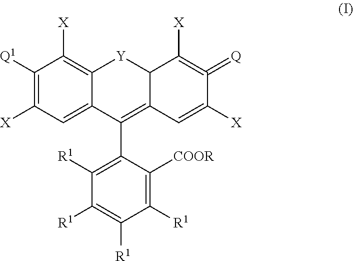

wherein each X is independently selected from H, Br, I, F, and Cl; Q is O or NRa; Q1 is OH, ORa, NHR^{a}, or NR^{a}₂; each R¹ is independently selected from H, Cl, F, I, and Br; Y is O, S, or Si(R^{a})₂; R is H or a cation; and each R^{a} is independently selected from C₁-C₁₂ alkyl; and wherein the photoreactive probe includes a photoreactive group coupled to a second moiety.

The disclosure further provides methods of proximity labeling proteins on a cell surface, the methods including admixing a cell having a surface membrane, the surface membrane having a transmembrane target protein and the cell having one or more further proteins, wherein the transmembrane target protein is coupled to an antibody-photocatalyst conjugate; and a photoreactive probe to form a mixture; and irradiating the mixture with light having a wavelength in a range of about 410 nm to about 570 nm to thereby label at least a portion of the one or more further proteins with the photoreactive probe and provide labeled proteins; wherein the photocatalyst has a structure according to formula (I).

Further aspects and advantages will be apparent to those of ordinary skill in the art from a review of the following detailed description. While the compositions and methods are susceptible of embodiments in various forms, the description hereafter includes specific embodiments with the understanding that the disclosure is illustrative, and is not intended to limit the disclosure to the specific embodiments described herein.

### BRIEF DESCRIPTION OF THE DRAWINGS

While the specification concludes with claims particularly pointing out and distinctly claiming the subject matter which is regarded as forming the present invention, it is believed that the invention will be better understood from the following description taken in conjunction with the accompanying drawings.

FIG. 1A shows a schematic of the MultiMap workflow. A photocatalyst, EY, is conjugated to an antibody that binds the target of interest (e.g. Fab arm of Ctx bound to the EGFR extracellular domain). Upon illumination, proteins are biotinylated, captured and digested for mass spectroscopy (MS) analysis. Proteomics hits are further examined by immunoprecipitation and predictive structural analysis via AlphaFold-Multimer. MultiMap is a useful platform for profiling local membrane protein interactomes both on live cells and between cell-cell synapses.

FIG. 1B is a WB showing EY-mediated photocatalytic biotinylation of BSA by a diazirine-biotin probe upon blue LED illumination. Biotinylation can be controlled temporally by pulsed light.

FIG. 1C shows EY triggers labeling of BSA with all four photo-probes (diazirine-biotin, aryl-azide-biotin, biocytin-hydrazide, and phenol-biotin).

FIG. 2 is a schematic showing current proximity labeling methods using one photocatalyst to activate one photoreactive probe.

FIG. 3 is a schematic showing a single photocatalyst of the disclosure that can activate multiple photoreactive probes.

FIG. 4A shows a synthetic scheme of Ctx-EY via a two-step bioconjugation workflow. An azido functionality was first introduced onto either Lys or Met residues using NHS or oxaziridine chemistry, respectively, followed by bio-orthogonal click reaction to couple EY.

FIG. 4B shows a schematic design to test intra- and inter-biotinylation of Ctx-EY and EGFR with or without EGF competition.

FIG. 4C shows targeted EGFR biotinylation with the diazirine-biotin photo-probe when triggered by either Ctx-NHS-EY (Ctx-EY) or Ctx-Ox-EY in vitro. Both conjugates selectively label EGFR in a light-dependent fashion, which is competed off by exogenous EGF.

FIG. 4D shows that EGFR is biotinylated by all three photo-probes: diazirine-biotin, aryl-azide-biotin or phenol-biotin using Ctx-EY.

FIG. 4E is a schematic showing biotinylation sites of diazirine-biotin (yellow), aryl-azide-biotin (cyan), biocytin-hydrazide (purple) and phenol-biotin (maroon) highlighted on the crystal structure of the EGFR ECD (grey) in complex with Ctx Fab (blue).

FIG. 5A is a schematic showing general on-cell labeling workflow using Ctx-EY conjugate and detection of biotin labeling using fluorescent streptavidin-AF647.

FIG. 5B shows a cellular binding assay of 100 nM Ctx, Ctx-EY, or Ctx-Ir conjugates on A431 cells via flow cytometry analysis, demonstrating similar on-cell binding.

FIG. 5C shows quantitative on-cell binding with diazirine-, aryl-azide- and phenol-biotin triggered by 100 nM Ctx-EY on A431 cells via flow cytometry analysis.

FIG. 5D shows quantitative on-cell labeling with diazirine-, aryl-azide- and phenol-biotin triggered by 100 nM Ctx-EY on A431 cells via flow cytometry analysis.

FIG. 5E is confocal microscopy imaging of antibody binding and on-cell biotinylation of Ctx-EY on A431 cells shows labeling mostly confined to cell surface. Scale bar=20 μm.

FIG. 6A is a schematic of a general proteomics workflow of interactome profiling using a Ctx-EY conjugate with or without EGF competition.

FIG. 6B is a Western blot showing biotinylation using the diazirine-biotin photo-probe on A431 cells using Ctx-EY in the absence and presence of EGF competition.

FIG. 6C is a volcano plot of Ctx-EY-mediated labeling of EGFR with or without EGF on A431 cells using diazirine-biotin. 41 significantly enriched proteins (log 2(ratio)≥1, p-value <0.05, unique peptide ≥2, n=3) are shown in the plot.

FIG. 6D shows the validation of six top protein hits using biotin-IP blots. Five were selectively enriched in a separate EGFR co-IP experiment.

FIG. 7A shows volcano plots of Ctx-EY mediated EGFR interactome profiling on A549 cells using three different photo-probes (biotin-diazirine, aryl-azide-biotin, or phenol-biotin, respectively, n=3). Significantly enriched proteins (log 2(ratio)≥1, p-value <0.05, unique peptide ≥2) are shown in the plots.

FIG. 7B shows a Venn diagram of EGFR interactome enriched from A431 cells using different photo-probes (biotin-diazirine (72), aryl-azide-biotin (188), and phenol-biotin (188)).

FIG. 7C shows enrichment ratios and validation of protein hits using all three photo-probes (biotin-diazirine, aryl-azide-biotin, and phenol-biotin).

FIG. 7D shows enrichment ratios and validation of protein hits from only the diazirine-biotin dataset.

FIG. 7E shows enrichment ratios and validation of protein hits from both aryl-azide-biotin and/or phenol-biotin datasets.

FIG. 7F shows AlphaFold-Multimer predictions of EGFR complexes which confirmed the direct interactions of EGFR with interactors found via MultiMap. EGFR ECD or ICD (blue) is shown in complex with the corresponding interactor protein (yellow for the ones interacting with EGFR ECD, green for the ones interacting with EGFR ICD) along with the pDockQ scores and BSASA.

FIG. 8A is a schematic of on-cell labeling of the T-cell synapse using a bispecific T cell engager (BiTE) that recognizes EGFR. Jurkat NFAT-GFP and HEK293T-Flag-EGFR were co-cultured in the presence of the BiTE before MultiMap was performed using an EY-conjugated α-Flag nanobody (α-Flag-EY). Cell-cell engagement was monitored by NFAT-GFP reporter gene activation. Photocatalytic labeling was characterized by flow cytometry before biotin-enriched proteins were analyzed by WB.

FIG. 8B is a schematic of target biotinylation of CDCP1 and CD3 at the T cell synapse using a bispecific T cell engager (BiTE) that recognizes CDCP1. Longer labeling radius using phenol-biotin was necessary for trans-labeling on Jurkat NFAT-GFP.

FIG. 8C is a volcano plot of proteins biotinylated on HEK-Flag-CDCP1 (cis-labeling) and Jurkat NFAT-GFP (trans-labeling) using phenol-biotin (n=3). Significantly enriched proteins from HEK-Flag-CDCP1 (log 2(ratio)≤−1, p-value <0.05, unique peptide ≥2) or Jurkat NFAT-GFP (log₂(ratio)≥1, p-value <0.05, unique peptide ≥2) are shaded in blue and red, respectively. Proteins known to associate with CDCP1 in STRING analysis are highlighted in red and those associated with CD3 complex in blue.

FIG. 8D is a schematic of on-cell labeling of α-CD19 chimeric antigen receptor (CAR)T cell system.

FIG. 8E shows (CAR)T cell-mediated trans-labeling of K562 cancer cells using all photo-probes (biotin-diazirine, aryl-azide-biotin, and phenol-biotin).

FIG. 8F shows target biotinylation of CD3 and CD19 at the CAR-T synapses using WB and MS analysis. Cells were sorted to differentiate cis- and trans-labeling before biotinylated proteins were enriched for analysis. Both phenol-biotin and aryl-azide-biotin enabled trans-labeling. Volcano plot of proteins biotinylated on Jurkat-CAR (cis-labeling) and K562-CD19 (trans-labeling) using phenol-biotin (n=3). Significantly enriched proteins from Jurkat-CAR (log 2(ratio)≤−1, p-value <0.05, unique peptide ≥2) or K562-CD19 (log 2(ratio)≥1, p-value <0.05, unique peptide ≥2) are shaded in blue and red, respectively. Proteins known to associate with the CAR complex in STRING analysis are highlighted in blue and those associated with CD19 in red.

FIG. 9 is a schematic showing the identification of Eosin Y (EY) as an organic photocatalyst.

FIG. 10 is a schematic showing targeted biotinylation induced by an antibody-EY conjugate.

FIG. 11 is a schematic of multi-scale interactome profiling in live cells.

### DESCRIPTION

Cell surface proteins are critical mediators of information, nutrients, and functions on cells and between them. The extracellular proteome, both secreted and membrane-bound, is encoded by more than 25% of the human genome. Proteomics methods have made great strides in characterizing the composition of the surface proteome in health and disease models. However, much less is known about the protein-protein interactions formed on the cell membrane, especially transient interactions that regulate cell signaling networks.

Proximity labeling proteomics (PLP) methods have enabled the identification of protein interactomes in complex cellular environments. These methods typically generate a single reactive intermediate locally to label and profile nearby proteins using imaging or proteomics. The first generation of PLP methods used genetically encoded enzymes such as APEX, BioID or TurboID to produce phenoxyl radicals or activated AMP that have long reactive half-lives (t_{1/2}>100 μsec). These methods are well-suited for characterizing cell-cell and organelle-specific interactomes given their long labeling range up to 3000 Å by labeling electron-rich amino acids. Singlet oxygen generators (SOG) triggers selective labeling on His at a shorter range given the shorter half-life of singlet oxygen in water (˜2-4 μs). However, proteins are estimated to be separated by only 60-70 Å on the crowded cell surface, thus making it challenging to identify the most proximal protein neighbors using long-range PLP approaches.

Most recently, PLP methods of very short range have emerged, enabling higher resolution mapping including μMap. These designs employ transition-metal or other photocatalysts attached to antibodies to trigger reactive intermediate with shorter half-lives such as carbenes or nitrenes (t_{1/2}˜2 and 10 ns, respectively). Activation of these probes enables labeling proteins at a significantly shorter range of ˜100-700 Å as well as broader amino acid coverage, thus making it much more appropriate for nearest neighborhood analysis. Collectively, the suite of PLP methods can cover a broad length scale for labeling protein neighborhoods and synapses but require multiple photocatalysts for adjustable resolution.

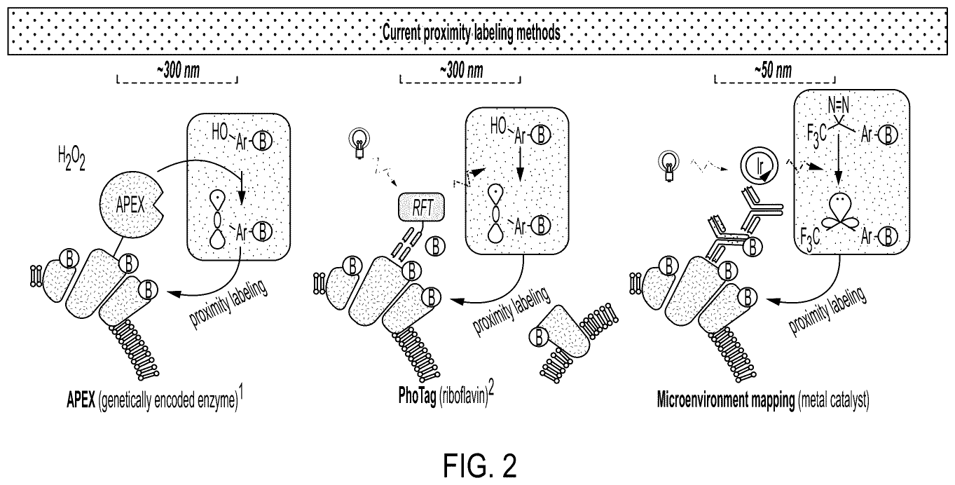

Existing proximity labeling techniques for investigating the interactions of membrane proteins are hindered by their restricted labeling radii. As shown in FIG. 2, current proximity labeling methods use one photocatalyst to activate one photoreactive probe. In contrast, the technique of the disclosure can provide a versatile and easily accessible platform capable of revealing interactomes across a multitude of spatial resolutions. By elucidating a comprehensive interactome, membrane protein functions can be better understood, leading to future therapeutic development endeavors.

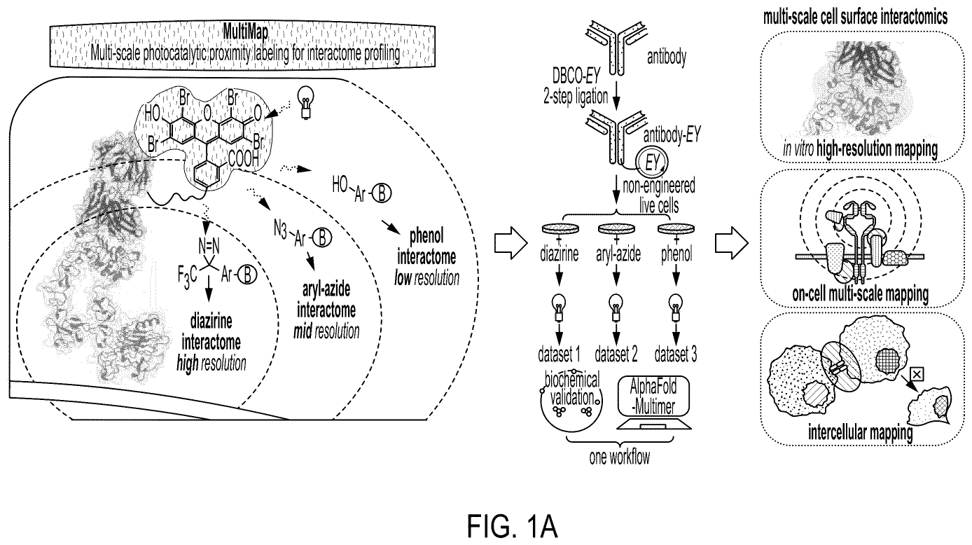

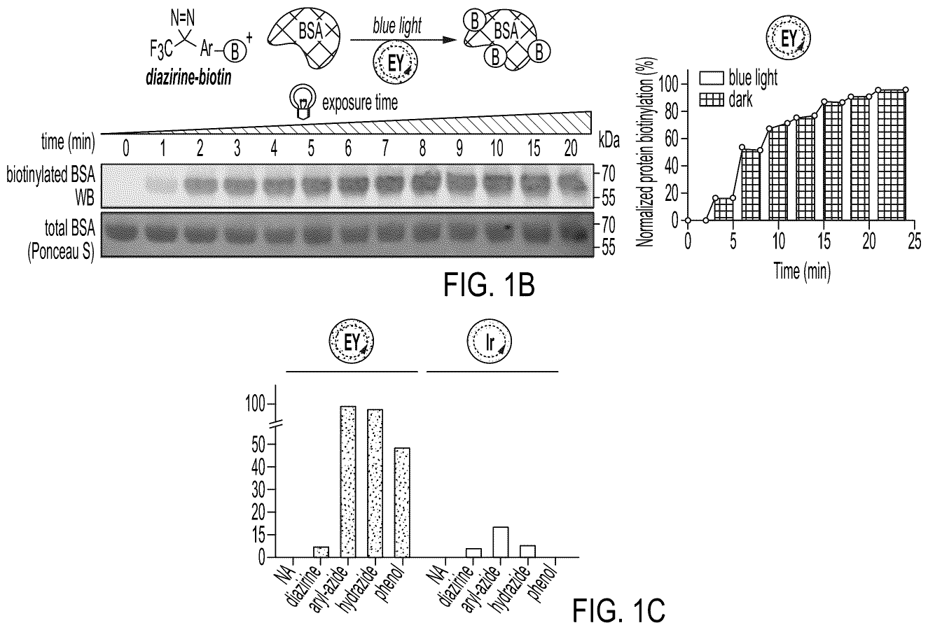

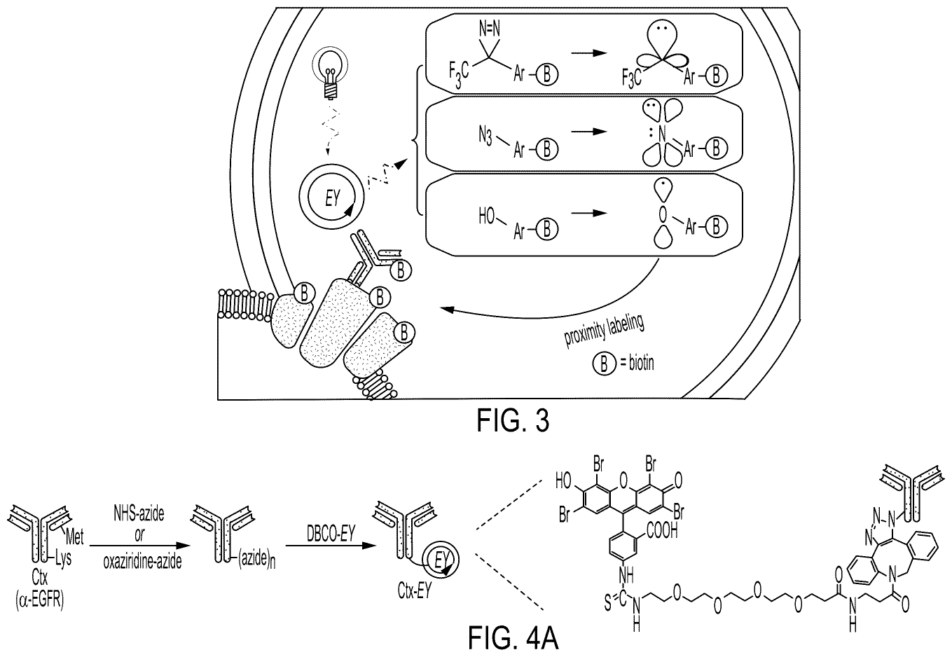

Provided herein is a first-in-class, multi-scale photocatalytic PLP technology, termed MultiMap (FIG. 1A) that allows short-, intermediate-, and long-range labeling from a single photocatalyst. The disclosure advantageously provides a single photocatalyst that can activate multiple photoreactive probes (FIG. 3). Advantageously, the single catalyst can trigger the activation of three distinct classes of reactive labeling reagents, each with a unique half-life, leading to labeling across multiple radii. By conjugating antibodies with this catalyst, interactomes associated with diseased-associated targets in live cells and across multiple length scales have been discovered. Using this this transformative technology additional binding partners for EGFR (Epidermal Growth Factor Receptor) have been revealed. The successful demonstration of this platform highlights its potential for identifying therapeutically relevant targets.

The disclosure provides a method of labeling a protein with a photoreactive probe comprising admixing a protein, a photoreactive probe, and a photocatalyst to form a mixture and irradiating the mixture with light having a wavelength in a range of about 410 nm to about 570 nm, wherein the photocatalyst has a structure according to formula (I):

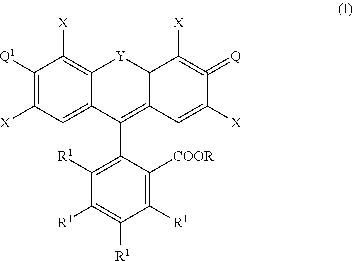

wherein each X is independently selected from H, Br, I, F, and Cl; Q is O or NR^{a}; Q¹ is OH, OR^{a}, NHR^{a}, or NR^{a}₂; each R¹ is independently selected from H, Cl, F, I, and Br; Y is O, S, or Si(R^{a})₂; R is H or a cation; and each R^{a} is independently selected from C₁-C₁₂ alkyl; and wherein the photoreactive probe comprises a photoreactive group coupled to a second moiety.

In the photocatalyst having a structure according to formula (I), R can be H. In the photocatalyst having a structure according to formula (I), R can be a cation. R can be an inorganic cation or an organic cation. R can be sodium, lithium, potassium, rubidium, or cesium.

In the photocatalyst having a structure according to formula (I), each X is independently selected from H, Br, I, F, and Cl. At least one X can be H, Br or I. All X can be Br or I. All X can be Br. All X can be I. All X can be H.

In the photocatalyst having a structure according to formula (I), Y can be O, S, or Si(R^{a})₂, wherein each R^{a} is independently selected from C₁-C₁₂ alkyl. Y can be O. Y can be S. Y can be Si(R^{a})₂. Y can be Si(R^{a})₂, wherein each R^{a} is independently selected from C₁-C₆ alkyl, for example, C₁ alkyl, C₂ alkyl, C₃ alkyl, C₄ alkyl, C₅ alkyl, or C₆ alkyl. Y can be Si(CH₃)₂. Y can be Si(CH₂CH₃)₂.

The term “alkyl” or “alkyl group” refers to a monoradical branched or unbranched saturated hydrocarbon chain having 1 to 40 carbon atoms. This term is exemplified by groups such as methyl, ethyl, n-propyl, isopropyl, n-butyl, isobutyl, t-butyl, pentyl, n-hexyl, n-heptyl, n-octyl, n-decyl, or tetradecyl, and the like. The term Cn means the alkyl group has “n” carbon atoms. For example, C₄ alkyl refers to an alkyl group that has 4 carbon atoms. C₁-C₁₂ alkyl refers to an alkyl group having a number of carbon atoms encompassing the entire range (i.e., 1 to 12 carbon atoms), as well as all subgroups (e.g., 1-11, 2-12, 3-10, 5-8, 1, 2, 3, 4, 5, 6, 7, 8, 9, 10, 11, and 12 carbon atoms). The alkyl group can be substituted or unsubstituted.

In the photocatalyst having a structure according to formula (I), Q can be O or NR^{a}. Q can be O. Q can be NR^{a}, wherein R^{a} can be C₁-C₁₂ alkyl. Q can be NR^{a}, wherein R^{a} can be C₁-C₆ alkyl. Q can be NR^{a}, wherein R^{a} can be C₁ alkyl. Q can be NR^{a}, wherein R^{a} can be C₂ alkyl.

In the photocatalyst having a structure according to formula (I), Q¹ can be OH, OR^{a}, NHR^{a}, or NR^{a}₂. Q¹ can be OH. Q can be O and Q¹ can be OH. Q can be NR^{a} and Q¹ can be OH. Q¹ can be OR^{a}, wherein R^{a} can be C₁-C₁₂ alkyl, C₁-C₆ alkyl, for example, C₁ alkyl, C₂ alkyl, C₃ alkyl, C₄ alkyl, C₅ alkyl, or C₆ alkyl. Q can be O and Q¹ can be OR^{a}. Q can be NR^{a} and Q¹ can be OR^{a}. Q¹ can be NHR^{a}, wherein R^{a} can be C₁-C₁₂ alkyl, C₁-C₆ alkyl, for example, C₁ alkyl, C₂ alkyl, C₃ alkyl, C₄ alkyl, C₅ alkyl, or C₆ alkyl. Q can be O and Q¹ can be NHR^{a}. Q can be NR^{a} and Q¹ can be NHR^{a}. Q¹ can be NR^{a}₂, wherein each R^{a} is independently C₁-C₁₂ alkyl, C₁-C₆ alkyl, for example, C₁ alkyl, C₂ alkyl, C₃ alkyl, C₄ alkyl, C₅ alkyl, or C₆ alkyl. Q can be O and Q¹ can be NR^{a}₂, wherein each R^{a}₂ is independently C₁-C₁₂ alkyl. Q can be O and Q¹ can be NR^{a}₂, wherein both R^{a}₂ are the same C₁-C₁₂ alkyl. Q can be NR^{a} and Q¹ can be NR^{a}₂, wherein each R^{a}₂ of the NR^{a}₂ is independently C₁-C₁₂ alkyl. Q can be NR^{a} and Q¹ can be NR^{a}₂, wherein both R^{a}₂ of the NR^{a}₂ are the same C₁-C₁₂ alkyl.

In the photocatalyst having a structure according to formula (I), each R¹ can be independently selected from H, Cl, F, I, and Br. At least one R¹ can be H. At least two R¹ can be H. At least three R¹ can be H. All R¹ can be H. At least one R¹ can be Cl. At least two R¹ can be Cl. At least three R¹ can be Cl. All R¹ can be Cl. At least one R¹ can be I. At least two R¹ can be I. At least three R¹ can be I. All R¹ can be I. At least one R¹ can be F. At least two R¹ can be F. At least three R¹ can be F. All R¹ can be F. At least one R¹ can be Br. At least two R¹ can be Br. At least three R¹ can be Br. All R¹ can be Br.

The photocatalyst can be selected from the group of:

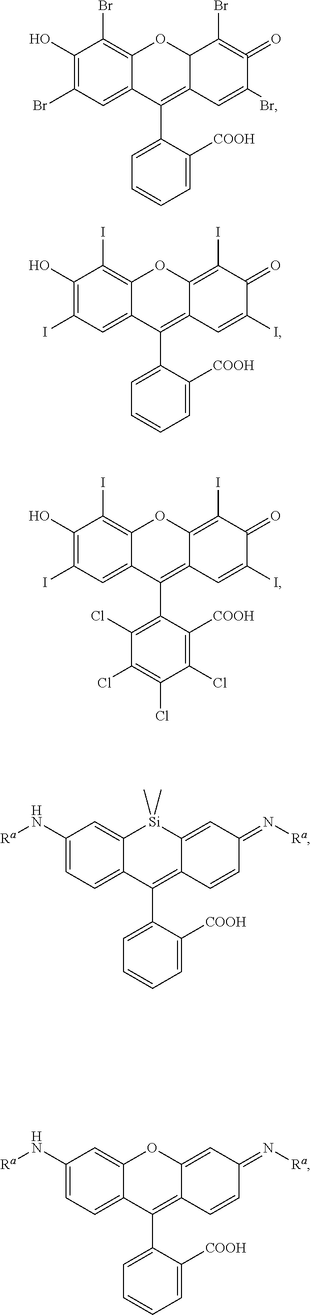

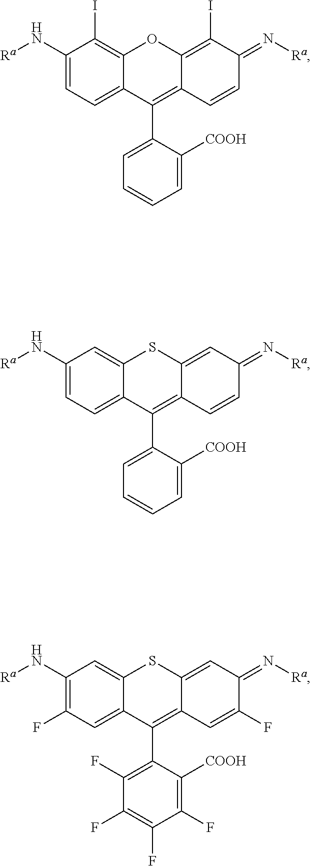

and salts thereof.

The photocatalyst can be Eosin Y (EY), a fluorescent dye commonly used in food chemistry and biological staining. EY can efficiently trigger labeling using diazirine, aryl-azide and phenol photo-probes with bio-compatible blue or green light. MultiMap was applied to profile high-resolution neighborhoods of the oncogenic epidermal growth factor receptor (EGFR) in different cellular contexts. More than 20 neighbors were identified and further validated their interactions via immunoprecipitation and in silico prediction models using AlphaFold-Multimer. MultiMap can capture long-range intercellular engagements between cancer cells and T lymphocytes induced by bi-specific T-cell engagers (BiTEs) and engineered chimeric antigen receptors (CARs). As shown in the Examples herein, MultiMap is an effective multi-scale PLP technology that can characterize local and distal cellular interactomes from a single photocatalyst. Without intending to be bound by theory, it is believed that due to the structural similarities between Eosin Y and the photocatalysts having a structure according to formula (I), the photocatalysts having a structure according to formula (I) will similarly efficiently allow short-, intermediate-, and long-range labeling from a single photocatalyst.

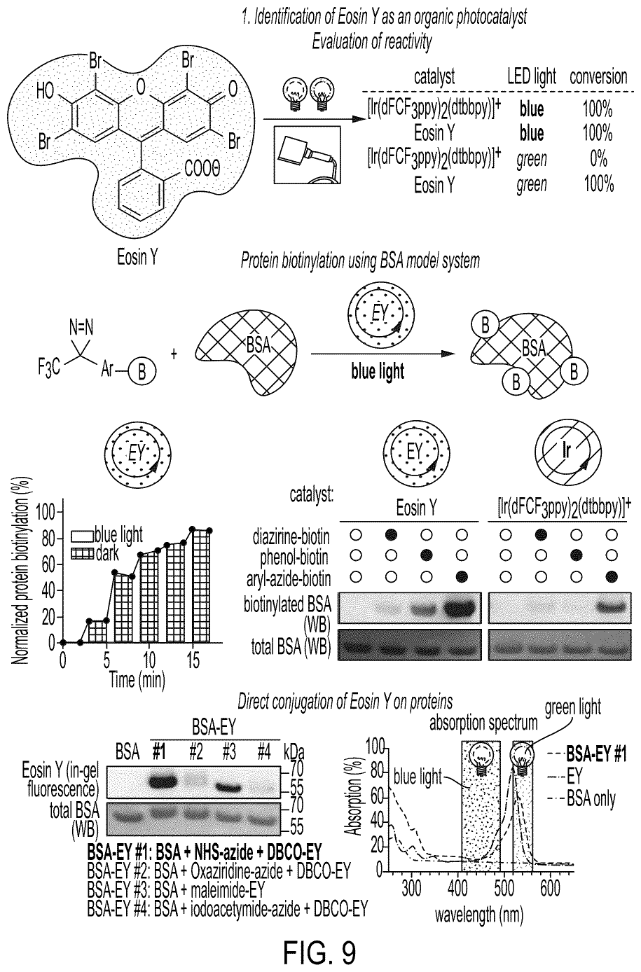

FIG. 9 describes the identification of Eosin Y as an organic photocatalyst. The reactivity of Eosin Y, a xanthane-based fluorescent dye, was evaluated. It was observed that Eosin Y can induce the activation of diazirine-biotin using green and blue LED light in vials. In a light-dependent manner, Eosin Y was shown to activate biotinylation on bovine serum albumin using diazirine-biotin. Remarkably, Eosin Y could activate three types of reactive probes utilized in the proximity labeling techniques, namely diazirine, aryl-azide, and phenol. The direct conjugation of Eosin Y onto BSA did not affect the photochemical properties of Eosin Y.

The protein that can be labeled can generally be any protein in proximity to the photocatalyst, for example, in proximity to a transmembrane protein having a photocatalyst coupled thereto, i.e., a “neighbor” to a transmembrane protein having the photocatalyst coupled thereto. The neighbor protein can be an intracellular protein or a transmembrane protein. The protein is generally considered to be in proximity to the photocatalyst/transmembrane protein if the protein is within labeling radius of the photocatalyst. The neighbor protein can b labeled if the protein is within about 100 Å to about 3000 Å from the photocatalyst. How proximal the protein can be from the photocatalyst depends on the photoreactive group of the photoreactive probe, as described herein.

The photoreactive probe can include one or more photoreactive group(s) coupled to a second moiety. The photoreactive group(s) can be selected from the group of a diazirine, an aryl-azide, a phenol, a hydrazide, and a combination thereof. The photoreactive groups are generally photoactive in response to light having a wavelength in a range of about 210 nm to about 300 nm. Accordingly, in the absence of a photocatalyst of the disclosure, when the photoreactive probe is irradiated with light having a wavelength of about 410 nm to about 570 nm, the photoreactive group is not activated by itself. In contrast, in the presence of the photocatalyst, when the mixture is irradiated with light having a wavelength of about 410 nm to about 570 nm, the photoreactive group is activated through the photocatalyst. and the second moiety of the photoreactive probe can be delivered to a protein in proximity of the photocatalyst.

As used herein, the term “coupled,” “coupling,” “couple,” and variations thereof encompass any one or more of covalent bond formation, hydrogen bond formation, ionic bond formation (e.g., electrostatic attraction), and van der Waals interactions, for example, through which the photoreactive group can adsorb to/adhere to/couple to/associate with a second moiety.

The second moiety can generally be any payload to be delivered to a protein of interest. The second moiety can be a label used to detect or enrich a protein, or provide a reactive handle to selectively introduce a further group to a protein. The second moiety can comprise biotin, a fluorophore, a crosslinking reagent, or a bioorthogonal handle such as an azide, an alkyne, a tetrazine, a dibenzocyclooctyne (DBCO), or a trans-cyclooctyne (TCO). Biotin and fluorophores can be used to label proteins for detection, for example. Crosslinking reagents and bioorthogonal handles can be used to introduce further groups (including but not limited to, a labeling reagent, an enzyme, a protein, or a bioactive molecule) to a protein of interest. The second moiety can be or comprise an enzyme, a peptide, a protein, or a bioactive molecule.

The photoreactive probe can be selected from the group of:

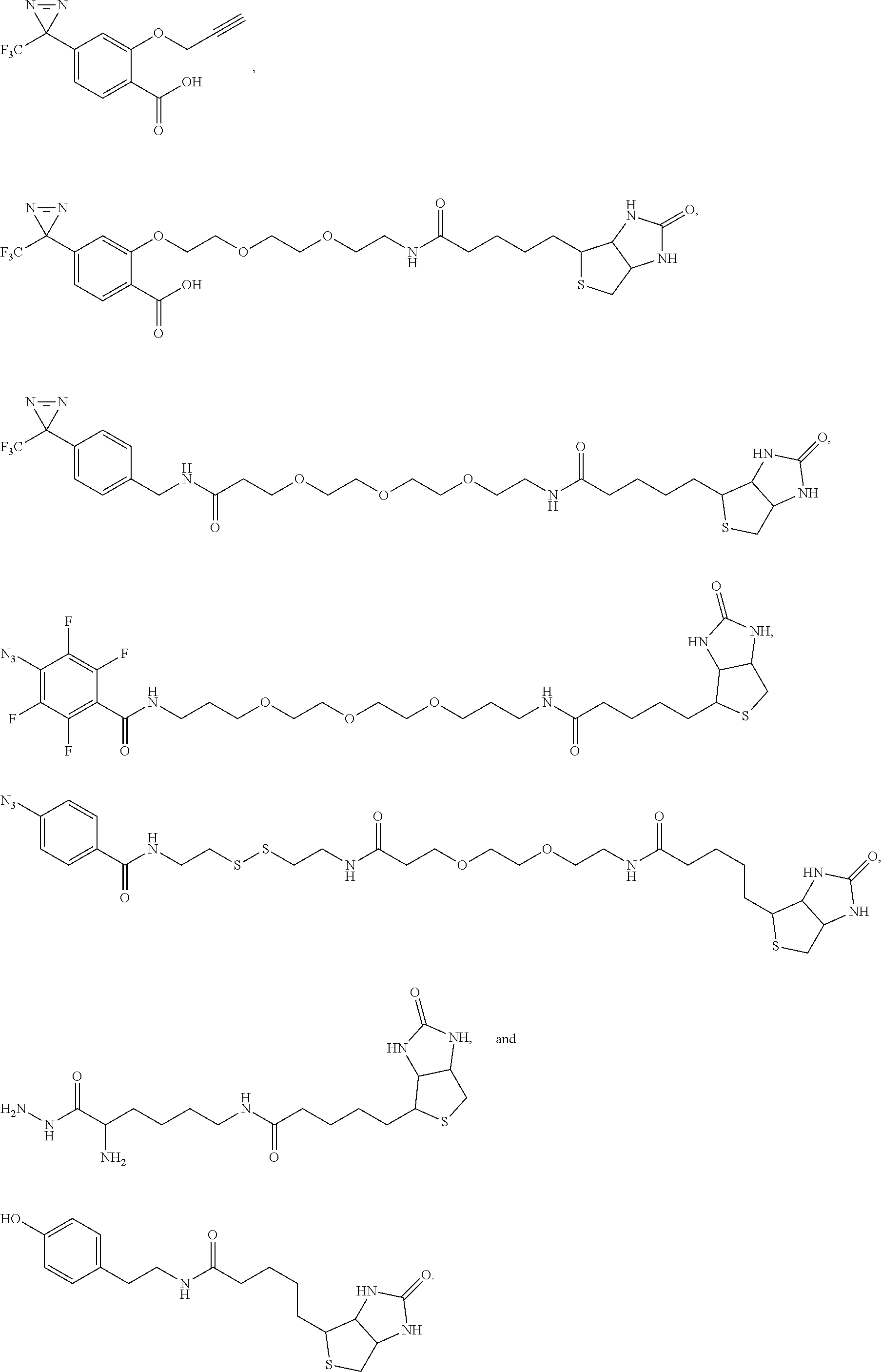

The photocatalyst can be conjugated to an antibody. The antibody is generally specific to a transmembrane target protein such that the antibody and the transmembrane target protein can bind to couple the photocatalyst to a cell including the transmembrane target protein. The photocatalyst can also be coupled to a tag binder that binds to an ecto-tag genetically engineered to the transmembrane target protein. Ecto-tag/binder pairs include, but are not limited to, spy tag/spycatcher, FLAG tag/anti-FLAG nanobody, green fluorescent protein (GFP) tag/anti-GFP nanobody, EPEA tag/anti-EPEA antibody, ALFA tag/anti-ALFA antibody, myc tag/anti-myc antibody, His tag/anti-His tag antibody, or HA/anti-HA antibody. In this way, the photocatalyst can be coupled through the tag/binder pair to the transmembrane target protein and, ultimately, to the cell. The photocatalyst can be coupled to the transmembrane target protein through a HaloTag ligand, wherein the transmembrane target protein comprises a HaloTag. The photocatalyst can be coupled to a HaloTag ligand to form a photocatalyst-HaloTag ligand conjugate, such as EY-HaloTag Ligand (EY-HTL), which has the chemical structure 2′,4′,5′,7′-tetrabromo-N-(2-(2-[(6-chlorohexyl)oxy]ethoxy)ethyl)-3′,6′-dihydroxy-3-oxo-3H-spiro[2-benzofuran-1,9′-xanthene]-6-carboxamide. The photocatalyst-HaloTag ligand conjugate, such as EY-HTL, can be synthesized by reacting 2′,4′,5′,7′-tetrabromo-3′,6′-dihydroxy-3-oxo-3H-spiro[2-benzofuran-1,9′-xanthene]-6-carboxylic acid with 1-(2-(2-aminoethoxy)ethoxy)-6-chlorohexane in the presence of a coupling reagent such as HATU (1-[Bis(dimethylamino)methylene]-1H-1,2,3-triazolo[4,5-b]pyridinium 3-oxide hexafluorophosphate) and a base such as DIPEA (N,N-Diisopropylethylamine) in a solvent such as DMF (N,N-Dimethylformamide). The reaction can be carried out at room temperature under an inert atmosphere. The product can be purified by chromatography, such as reverse phase chromatography.

The protein, photoreactive probe, and photocatalyst can generally be admixed under any conditions wherein the protein is stable. The protein can be provided as a transmembrane protein or an intracellular protein. The admixing can include combining the protein, the photoreactive probe, and the photocatalyst in solution. The solution can be an aqueous solution. The solution can include a buffer. The admixing can take place for any time and temperature that does not denature or otherwise destroy the protein. For example, the admixing can take place at a temperature in a range of about 4° C. to about 60° C., or about 4° C. to about 50° C., or about 4° C. to about 37° C., or about 20° C. to about 25° C., or about 4° C., about 20-25° C., or about 37° C. The duration of admixing can depend on the temperature of admixing. In general, as the temperature increases the duration of admixing can decrease as the reaction will proceed faster and the protein will generally be less stable at higher temperatures.

The disclosure further provides a method of proximity labeling proteins on a cell surface, the method including (a) admixing a cell having a surface membrane, the surface-membrane having a transmembrane target protein and the cell having one or more further proteins, wherein the transmembrane-target protein is coupled to an antibody-photocatalyst conjugate; and a photoreactive probe to form a mixture; and (b) irradiating the mixture with light having a wavelength in a range of about 410 nm to about 570 nm to thereby label at least a portion of the one or more further proteins with the photoreactive probe and provide labeled proteins; wherein the photocatalyst has a structure according to formula (I), as described herein. The method can optionally include a washout procedure to remove unbound photocatalyst or photocatalyst conjugate from the cells. For example, when using a photocatalyst-HaloTag ligand conjugate such as EY-HTL, the cells can be washed multiple times with fresh media to remove free photocatalyst-HaloTag ligand. In some cases, the cells can be washed four times with fresh media including incubation periods between washes, such as about 15 min soaks between washes. A fluorescent analog, such as JF646-HTL (Janelia Fluor-646 with HaloTag Ligand), can be used to validate the removal of unbound ligand by flow cytometry.

The transmembrane target protein can generally be any protein that is expressed extracellularly such that the antibody-photocatalyst conjugate can access the protein for binding. The transmembrane target protein can also include one or more ecto-tag/binder pair such as, for example, spy tag, FLAG tag, green fluorescent protein (GFP) tag, EPEA tag, ALFA tag, myc tag, His tag, Halo tag, or HA tag. The transmembrane target protein can include a first ecto-tag on the extracellular N-terminus of the transmembrane target protein and a second ecto-tag on the intracellular C-terminus. The first and second ecto-tags can be different ecto-tags. The transmembrane target protein can be directly coupled to the antibody-photocatalyst conjugate. The transmembrane target protein can be coupled to the antibody-photocatalyst conjugate through an ecto-tag, wherein the ecto-tag is directly coupled to the transmembrane target protein and the antibody of the antibody-photocatalyst conjugate is directly coupled to the ecto-tag. The transmembrane target protein can be epidermal growth factor (EGFR), CUB domain-containing protein 1 (CDCP1), B-lymphocyte antigen CD19 (CD19), human epidermal growth factor receptor 2 (HER2), immunoglobin E (IgE), or B-cell maturation antigen (BCMA). The cells can be any cell that expresses a transmembrane target protein of interest. The cells can be non-small cell lung cancer cells. The cells can be A549 cells, which are non-small cell lung cancer cells with physiologically relevant expression levels of EGFR. A549 cells may have an EGFR expression level of about nTPM=59.7 (normalized transcripts per million).

The one or more further proteins can generally be any proteins in proximity of the transmembrane target protein, i.e., that “neighbor” the transmembrane target protein. As used herein, a protein is “in proximity of the transmembrane target protein” if the protein is within energy transfer range of the photocatalyst coupled to the transmembrane target protein through the antibody. The one or more further proteins can be in a range of about 100 Å to about 3000 Å of the transmembrane target protein. As described herein, how proximal the one or more further protein is from the transmembrane target protein can depend on the photoreactive group of the photoreactive probe. The one or more further proteins can be a transmembrane protein, an intracellular protein, or a combination thereof. At least a portion of the neighbor proteins are labeled by the photoreactive probe upon irradiating the mixture with light having a wavelength in a range of about 410 nm to about 570 nm. Intracellular labeling using certain photoreactive probes, such as phenol-biotin, may be quenched by intracellular components. Glutathione can significantly decrease phenol-biotin-based labeling in a dose-dependent manner. Other intracellular components that may have quenching effects include superoxide dismutase-1 (SOD1), glutathione peroxidase (GPx), and catalase.

The antibody can generally be any antibody specific for binding to the transmembrane target protein. Antibodies specific for coupling to a transmembrane-target protein are generally known in the art. For example, the antibody can be an epidermal growth factor receptor inhibitor and the transmembrane-target protein can be an epidermal growth factor receptor. The antibody can be cetuximab and the transmembrane protein can be an epidermal growth factor receptor. The antibody can be trastuzumab and the transmembrane target protein can be HER2.

The photoreactive probe can be any photoreactive probe disclosed herein. The photoreactive probe can include a photoreactive group selected from the group of a diazirine, an aryl-azide, a phenol, a hydrazide, or a combination thereof. The photoreactive probe can include more than one photoreactive groups.

The mixture can include two or more photoreactive probes. The two or more photoreactive probes can be different from each other in that the photoreactive probes have different photoreactive groups. The two or more photoreactive probes can be added to the mixture concurrently. Alternatively, a first photoreactive probe can be added to the mixture and the mixture irradiated with light having a wavelength in a range of about 410 nm to about 570 nm to form a second mixture, followed by adding a second photoreactive probe to the second mixture and irradiating the second mixture with light having a wavelength in a range of about 410 nm to about 570 nm to form a third mixture. Additional photoreactive probes can be added concurrently with the first or second photoreactive probes and or in a continued step-wise manner. Once all photoreactive probes have been added followed by irradiation with light having a wavelength in a range of about 410 nm to about 570 nm, the method can further include characterizing the labeled proteins. The labeled proteins can be characterized by mass spectrometry, fluorescence imaging, DNA barcoding, or a combination thereof. The labeled proteins can be validated using orthogonal methods such as proximity ligation assay (PLA). PLA can provide spatial proximity information with high sensitivity and can be performed using imaging-based detection methods or flow cytometry-based detection methods.

The mixture can be irradiated with light having a wavelength in a range of about 410 nm to about 570 nm. The light can be a green light having a wavelength in a range of about 500 nm to about 570 nm. The light can be a blue light having a wavelength in the range of about 410 nm toa bout 470 nm.

The temporal resolution of the labeling can be controlled by the duration of light exposure. A short light exposure, e.g. about 2 min, can trigger ample protein labeling, allowing for high temporal resolution in tracking dynamic protein interactions. The labeling reactions may not continue after light activation when samples are placed in the dark, indicating that the reaction is light-dependent and that temporal resolution can be controlled by external light.

When the term “about” is used in describing a value or an end-point of a range, the disclosure should be understood to include the specific value or end-point referred to. When a numerical value or end-point does not recite “about,” the numerical value or end-point of a range is intended to include two embodiments: one modified by “about,” and one not modified by “about.” It will be further understood that the endpoints of each of the ranges are significant both in relation to the other endpoint, and independently of the other endpoint. The term “about” is used according to its ordinary meaning, for example, to mean approximately or around. In one embodiment, the term “about” means ±10% of a standard value or range of values. In another embodiment, the term “about” means ±5% of a standard value or range of values.

The cell and photoreactive probe can generally be admixed under any conditions wherein the proteins of the cell are stable. The admixing can include combining the cell and photoreactive probe in solution. The solution can be an aqueous solution. The solution can include a buffer. The admixing can take place for any time and temperature that does not denature or otherwise destroy the proteins of the cell. For example, the admixing can take place at a temperature in a range of about 4° C. to about 60° C., or about 4° C. to about 50° C., or about 4° C. to about 37° C., or about 20° C. to about 25° C., or about 4° C., about 20-25° C., or about 37° C. The duration of admixing can depend on the temperature of admixing. In general, as the temperature increases the duration of admixing can decrease as the reaction will proceed faster and the proteins will generally be less stable at higher temperatures.

The method can further comprise preparing a cell having a surface membrane, the surface membrane having a transmembrane target protein and the cell having one or more further proteins, wherein the transmembrane target protein Is coupled to an antibody-photocatalyst conjugate. The cell having a transmembrane target protein coupled to an antibody-photocatalyst conjugate can be prepared by admixing a cell with an antibody-photocatalyst conjugate under the general conditions described herein for all admixing steps. The method can further provide preparing an antibody-photocatalyst conjugate by admixing an antibody with a photocatalyst under the general conditions described herein for all admixing steps.

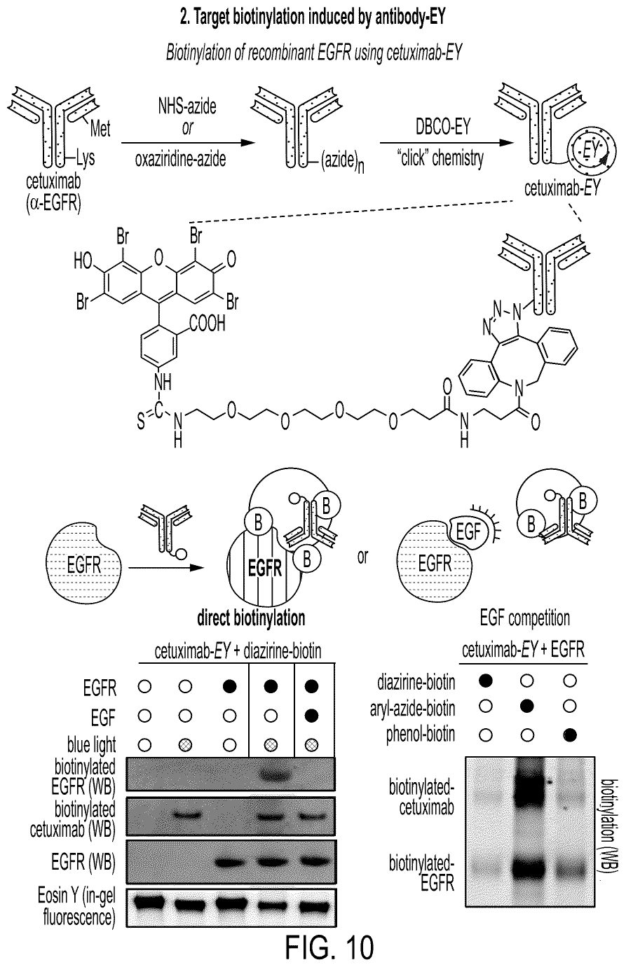

FIG. 10 describes targeted biotinylation induced by an antibody-Eosin Y conjugate, specifically, the biotinylation of recombinant EGFR using cetuximab-EY. EY was coupled to cetuximab, an FDA-approved monoclonal antibody targeting EGFR. The conjugated cetuximab-OEY could specifically recognize its target EGFR and biotinylate it upon blue light irradiation. The presence of EGF can selectively hinder the recognition of EGFR by cetuximab, thereby rescuing it from biotinylation.

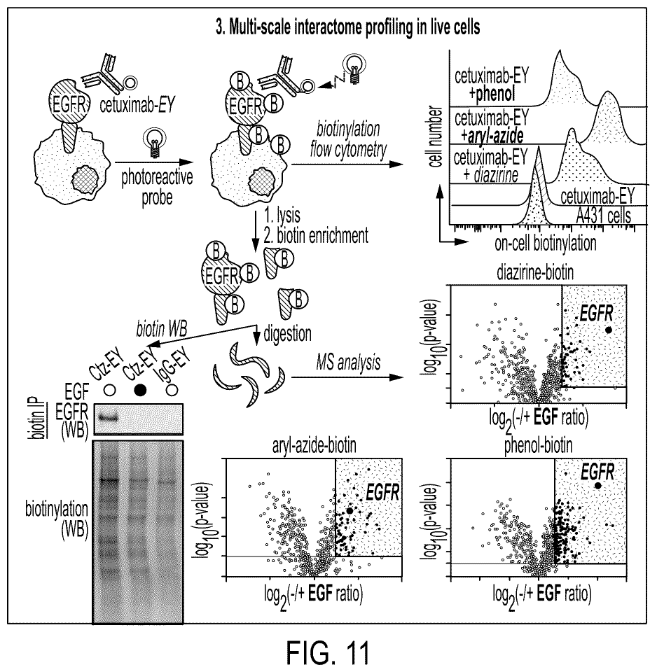

FIG. 11 describes multi-scale interactome profiling in live cells. Using cetuximab-EY, on-cell biotinylation, selective enrichment of EGFR, and profiling of the EGFR interactome was successfully achieved.

Demonstrated herein is a multi-scale PLP technology, MultiMap, that enables proximity labeling and interactome profiling with adjustable resolution. While the specific examples demonstrate the use of Eosin Y (EY) as the photocatalyst, the disclosure is not limited to EY. The photocatalyst EY was capable of triggering a broad range of photo-probes with different half-lives. It is commercially available, bio-compatible, and shown to be readily conjugated to seven different proteins and antibodies by commonly accessible methods. Simple targeting by EY-conjugated antibodies obviates the need for cell engineering. EY-mediated labeling is rapid and light-dependent, which potentially allows kinetic control of the labeling.

Coupled with standard biochemical validation and the recently developed AlphaFold-Multimer algorithm for structural prediction, the MultiMap proximity labeling proteomics workflow provides three orthogonal and integrated pillars for high-resolution profiling of protein neighborhoods. In addition to identification of new potential neighbors, many proteins known to functionally interact with EGFR that are reported to stabilize, modulate, or act as substrates for EGFR were identified. One of the most striking targets identified was the phosphatase, PTPRF, which could be a functional off-switch for EGFR. Interestingly, AlphaFold-Multimer predicts the ECD of PTPRF binds to the back side of the EGFR ECD away from the dimer interface, and that the ICD of the phosphatase binds to the intracellular kinase domain of EGFR. Despite the fact that EY-antibodies recognize extracellular targets, it was found that some of the high-confidence hits were intracellular proteins. Some are known to functionally associate with EGFR, for which high-confidence AlphaFold-Multimer binary models were constructed. It is possible that the labeling is caused by cell penetrance of the activated photo-probe when triggered by EY.

It is unlikely that all these identified neighbors bind simultaneously to EGFR. In fact, some proteins are predicted to bind over the same sites. These data suggest EGFR can be in multiple neighborhoods which are dynamic and may have multiple functions that are yet to be revealed. It is also important to note that the binder used in this study, Ctx, is an inhibitor of EGFR function. Thus, candidates identified in these studies are specifically from the EGFR off-state neighborhood. Without intending to be bound by theory, it is believed that MultiMap will be useful to study on-state, drug-bound, and resistance mutant neighborhoods, which will give a comprehensive map of the EGFR interactomes.

MultiMap was also effective for long-range labeling of cell-cell synapses. As shown for the ones activated by BiTE or (CAR)T, it was found that spatial variability among synaptic junctions can be addressed by using photo-probes with different labeling radii. The unique advantage of MultiMap allowing multi-scale labeling, potentiates its application for interactome profiling of additional intercellular interaction networks. Information of these networks will help deepen our understanding of the underlying mechanisms behind intercellular recognition and signaling. In cases where antibodies are not available, one can use a genetically encoded tag on the target ECD, similar to the Flag and myc ecto-tags introduced in this study. Proteome-wide interactome profiling for membrane proteins may also be done using these ecto-tags on par with the scale for the intracellular OpenCell system.

### EXAMPLES

### Activation of Photo-Probes for Protein Labeling

The ability of Eosin Y to label bovine serum albumin (BSA) using a diazirine-biotin probe in the presence of light was tested (FIG. 1B). Time- and light-dependent accumulation of biotinylated BSA was observed via Western blot (WB) analysis. Labeling plateaued within 6 minutes of blue LED illumination (FIG. 1B, left). A pulse-light experiment (FIG. 1B, right) demonstrated that the catalytic function of EY is light-dependent. Parallel comparison of EY-activated BSA biotinylation showed significantly higher signal than background with 2 min and 10 min illumination.

The photocatalytic labeling on BSA by WB analysis was tested and EY was found to efficiently catalyze biotin labeling in the presence of aryl-azide biotin, biocytin-hydrazide or phenol-biotin. The extents of BSA labeling using soluble EY among the four biotin-containing reactive probes, herein, also referred to as “photo-probes,” ranged in the following order: aryl-azide-biotin (>95%), biocytin-hydrazide (>90%), phenol-biotin (˜40%) and diazirine-biotin (˜5%) (FIG. 1C).

The absorption peak for EY (λₘₐₓ=517 nm) is significantly red-shifted compared with that of a known Ir catalyst (λₘₐₓ=420 nm). Without intending to be bound by theory, it is believed that the red-shift indicates that EY could be more bio-compatible given the potential toxicity of blue light. A closer examination of biotinylation efficiency in a time-course experiment demonstrated that EY indeed efficiently catalyzed labeling of BSA with green LED (λ=525 nm) while the Ir catalyst showed no labeling. More than 80% labeling of BSA was achieved upon 3 min green LED exposure of EY with all four photo-probes. It was also found that EY maintained its photocatalytic function above its pKa (pH=3.5) and thus is compatible with labeling across a wide range of physiologic pH conditions.

Conjugation of EY onto Proteins

Dibenzocyclooctyne (DBCO)-PEG4-EY was synthesized via an amine-isothiocyanate reacting according to Scheme 1, shown in the Examples, below. Different conjugation methods for EY onto BSA and antibodies were evaluated. Conjugation efficiency and stoichiometry for attaching a click-compatible azido functionality specifically to Lys, Met or Cys using N-hydroxy succinimide (NHS) ester, oxaziridine, or maleimide/iodoacetamide warheads, respectively, were evaluated. EY-conjugation via NHS-azide ligation produced the most efficient conjugation; conjugated EY also efficiently catalyzed BSA self-biotinylation with diazirine-biotin, aryl-azide-biotin and phenol-biotin.

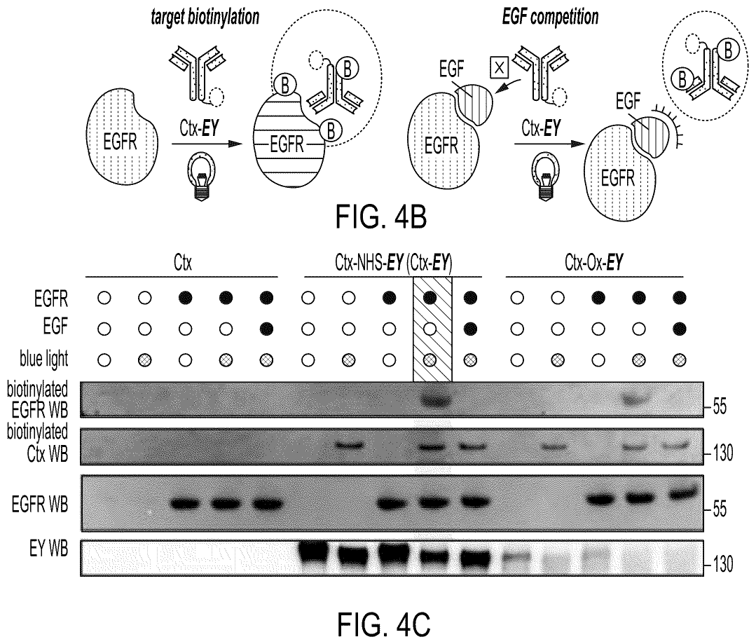

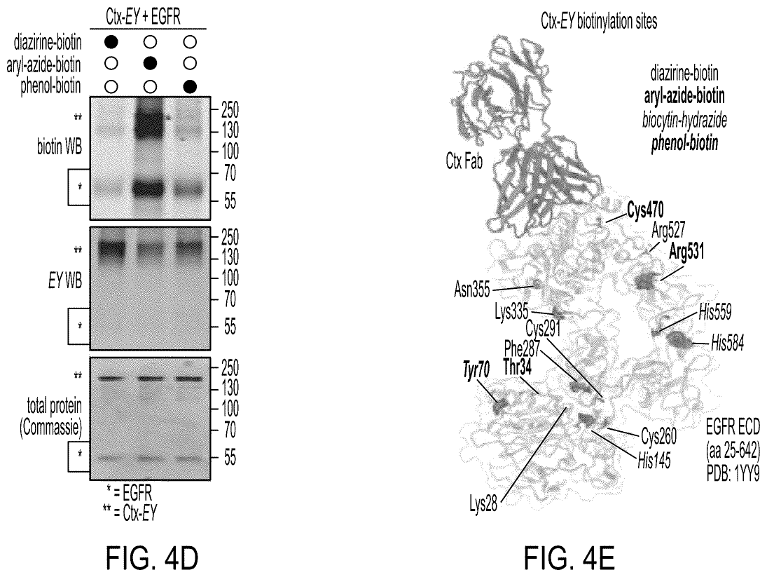

EY was conjugated to cetuximab (Ctx), an FDA-approved antibody that selectively binds EGFR and competes for epidermal growth factor (EGF) binding, thus turning-off EGFR signaling and cell proliferation in cancer (FIG. 4B). Ctx does not have Lys, Met or Cys residues in the CDRs or in the contact epitope with the EGFR ectodomain (ECD, aa 1-645, PDB: 1YY9), suggesting all bioconjugation methods are viable without impairing binding. The same panel of bioconjugation warheads were tested on Ctx, generating similar levels of conjugation as seen for BSA. Quantification of the levels of conjugation by WB analysis or EY absorption indicated that a stoichiometry of eight and two EY catalysts were installed per Ctx-NHS-EY and Ctx-Ox-EY, respectively.

Intra- and inter-molecular labeling of the Ctx-EY conjugates were tested with recombinant human EGFR ECD and in competition with EGF (FIG. 4C). Upon blue LED illumination, both Ctx-NHS-EY and Ctx-Ox-EY conjugates demonstrated self-labeling in a light-dependent manner. There was a higher degree of biotinylation with Ctx-NHS-EY which contains ˜4-fold more conjugated EY than Ctx-Ox-EY. Intermolecular EGFR labeling with both EY-conjugated constructs occurred in a light-dependent manner (FIG. 4C), indicating that the conjugation of EY did not interfere with Ctx binding to EGFR, as expected. Pre-incubation of EGF prevented labeling, demonstrating that direct binding is necessary for target labeling (FIG. 4C). The generality of the workflow was demonstrated by the same NHS and oxaziridine bioconjugation and labeling using a trastuzumab (Trz) Fab that binds the HER2 receptor ECD. Similar intermolecular labeling of HER2 was observed with Trz-NHS-EY or Trz-Ox-EY in a light-dependent manner. The demonstration of EGFR and HER2 labeling in vitro supports the broad applicability of the bioconjugation strategy and photo-probe labeling workflow.

The NHS-azide conjugate (abbreviated to Ctx-EY) was used to evaluate EGFR labeling efficiencies given its higher bioconjugation and photo-probe labeling efficiency. EGFR labeling efficiencies with diazirine-, aryl-azide-, and phenol-biotin photo-probes were evaluated in parallel (FIG. 4D). All three probes labeled the EGFR ECD to increasing levels: aryl-azide-biotin>phenol-biotin>diazirine-biotin. Without intending to be bound by theory, it is believed that the differing yields were a result of the combined effects of the reactive radical intermediates: half-lives (phenol>>aryl-azide>diazirine), yield of reaction with protein (phenol>aryl-azide>diazirine), and amino-acid preference observed (diazirine˜aryl-azide>>phenol).

The specific sites of biotinylation for self-labeling of BSA and binary complex labeling of Ctx and EGFR were explored with different photo-probes using MS analysis. For diazirine-biotin, 17 biotinylated sites on BSA were found, and 30 sites on the Ctx-EGFR complex were found, with good coverage of modified peptides over the light and heavy chains of Ctx, as well as EGFR ECD (FIG. 4E). The modification sites on BSA and Ctx-EGFR systems were further characterized for the other probes. As expected, phenol-biotin mostly labeled Tyr/Trp, while labeling with biocytin-hydrazide was found exclusively on His. Diazirine-biotin and aryl-azide-biotin showed very broad amino acid preference, consistent with previous reports.

### Ctx-EY Catalyzed Target Labeling of EGFR on Cells

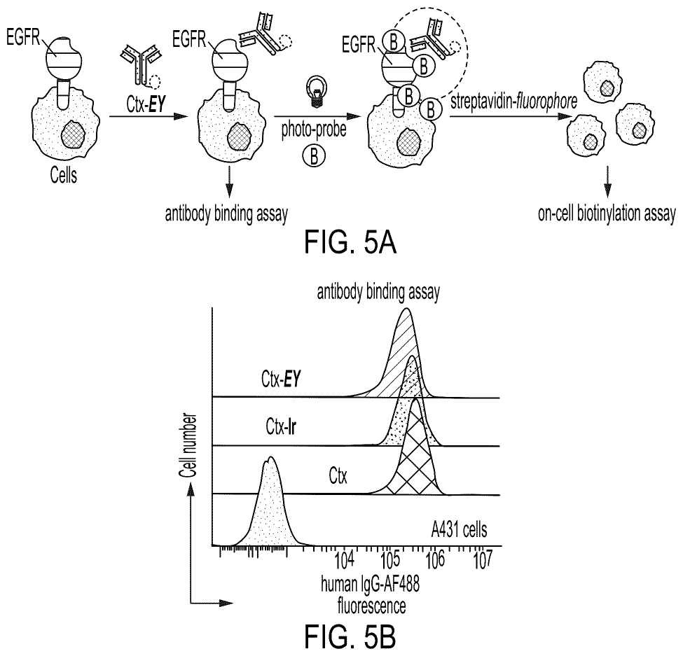

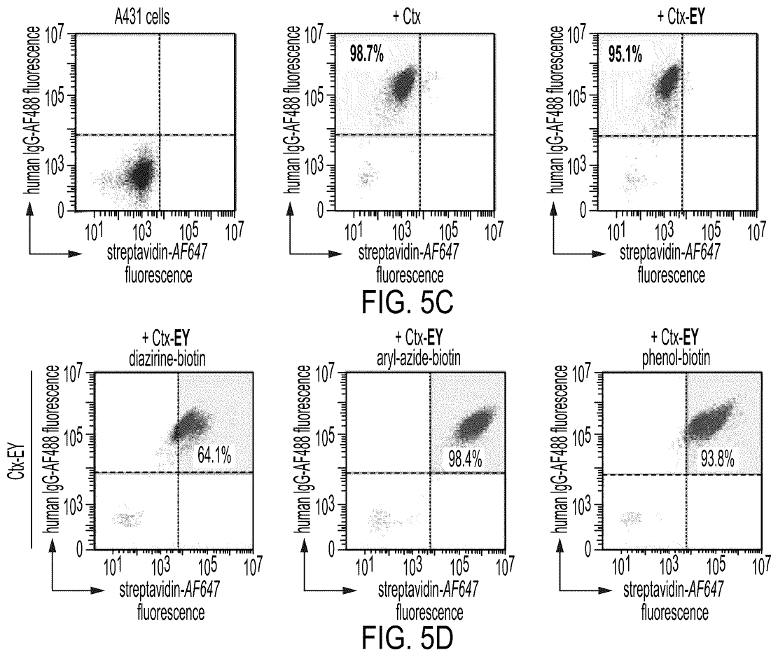

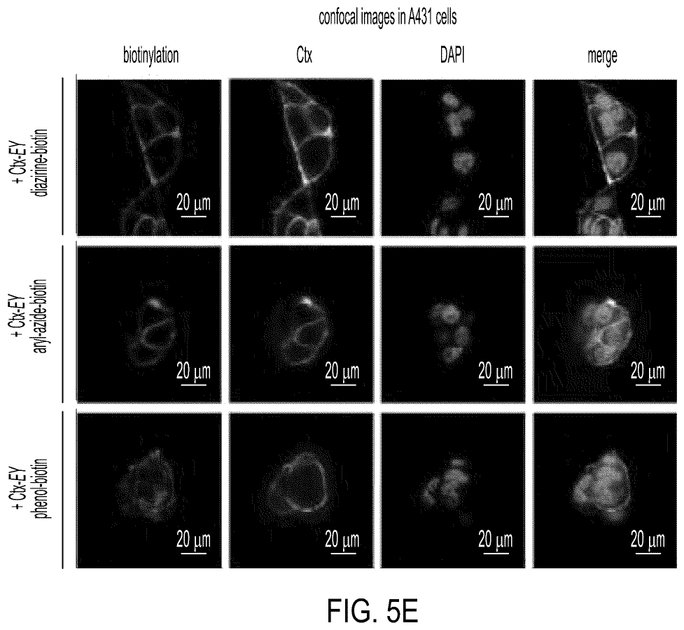

The ability of Ctx-EY to bind EGFR and label live cells was evaluated (FIG. 5A). First, the Ctx-EY conjugate was incubated with an epithelial skin cancer cell line, A431 cells, that endogenously expresses very high levels of wild-type EGFR (nTPM=2978). On-cell binding for the Ctx-conjugates, both Ctx-EY and Ctx-Ir, was confirmed via flow cytometry showing that the conjugation of EY or the Ir-catalyst did not affect binding (FIG. 5B). Detailed titration from 1 nM to 10 μM of Ctx and Ctx-EY analyzed via flow cytometry further confirmed conjugation did not detectably affect cell binding (FIG. 5B). A549 cells with more typical levels of EGFR (nTPM=59.7) and NCI-H441 cells with very low EGFR expression (nTPM=29.8), were also tested and both showed proportionally reduced binding of Ctx-EY and was similar to Ctx.

On-cell proximity labeling with diazirine-, aryl-azide- and phenol-biotin photo-probes upon blue LED illumination was performed (FIG. 5D). A range of Ctx-EY concentrations were tested and efficient biotinylation on cells at 100 nM was observed (FIG. 5D). The diazirine-biotin, aryl-azide biotin, and phenol-biotin labeling caused a major shift of biotinylation in the flow cytometry profile of 64%, 98%, and 94%, respectively in A431 cells (FIG. 5D). This is consistent with the order of labeling efficiencies observed in vitro. Similar patterns of biotinylation were observed in A549 and NCI-H441 cells, which were proportional to their EGFR expression levels. The Ctx-Ir only activated cell biotinylation with diazirine-biotin and aryl-azide-biotin and not phenol-biotin.

Cell biotinylation induced by Ctx-EY was visualized via confocal microscopy (FIG. 5E). The labeled A431, A549 and NCI-H441 cells were co-stained with both α-human IgG-AlexFluor488 and streptavidin-AlexaFluor647 to visualize Ctx and biotinylation, respectively. We confirmed that the Ctx-EY conjugate was located on the cell membrane. Biotinylation using the diazirine- and aryl-azide-biotin photo-probes were observed mainly on the cell membrane, whereas the phenol-biotin labeling was more diffuse, consistent with the longer half-life and labeling range of the phenoxyl radical.

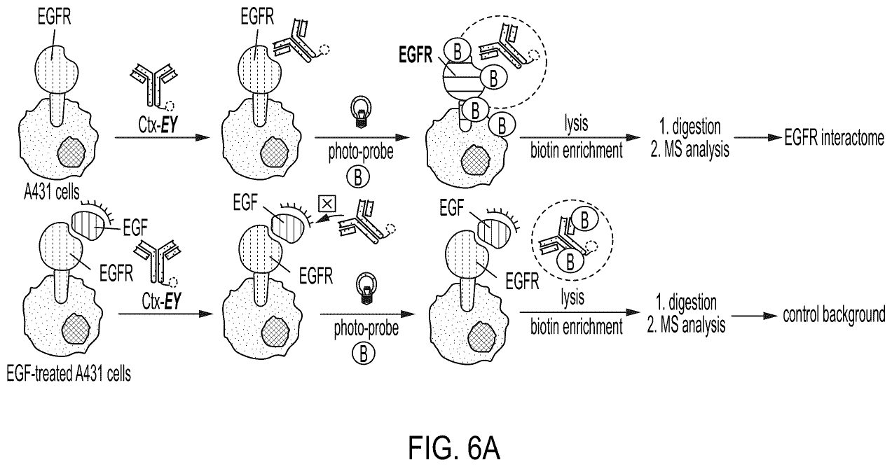

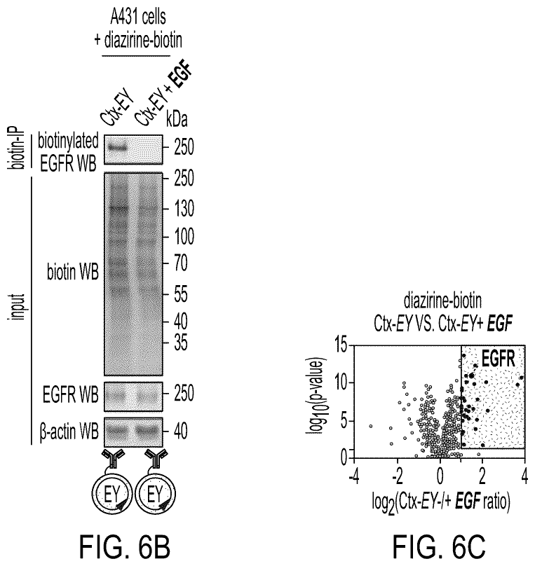

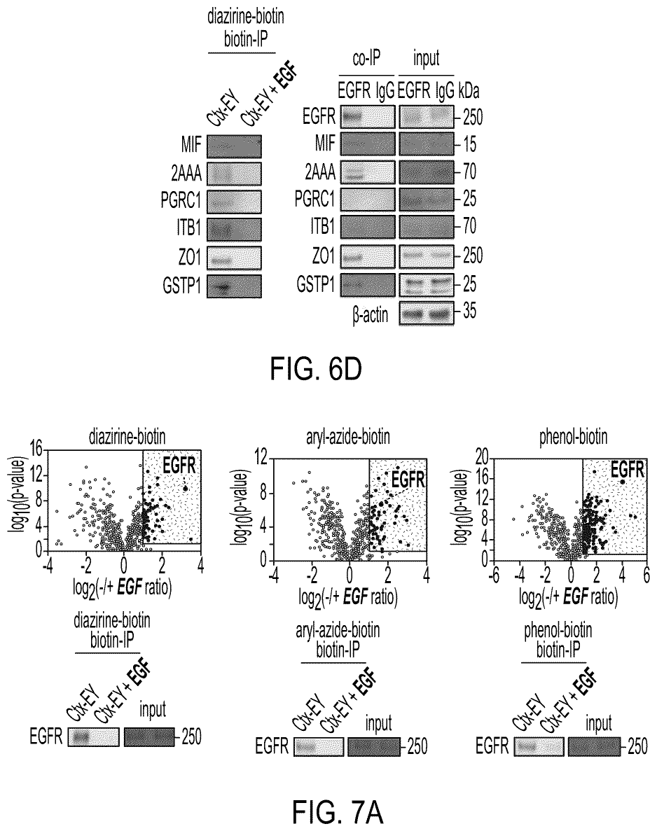

A proteomics workflow was developed to label the EGFR neighborhood (FIG. 6A), focusing first on A431 cells with highest levels of EGFR and using the most reactive diazirine-biotin photo-probe. A431 cells were incubated with or without EGF competition first and then performed the on-cell biotinylation workflow using Ctx-EY, followed by biotin enrichment using neutravidin beads. WB analysis confirmed selective biotinylation of EGFR which was ablated in the presence of EGF (FIG. 6B). Dose-dependent EGFR labeling over a wide range of Ctx-EY concentrations of 1-1000 nM was also observed, which was competed off by either EGF or unlabeled Ctx.

Cells were treated with Ctx-EY in the presence or absence of EGF competition and biotinylated proteins were captured on neutravidin beads and digested on-bead with trypsin. Samples were prepared in biological triplicate for MS analysis using label-free quantitation (volcano plot shown in FIG. 6C). A total of 536 proteins were identified, with 41 proteins enriched by more than two-fold with Ctx-EY relative to EGF competition (log 2(ratio)≥1, p-value <0.05, unique peptide ≥2). EGFR was among the highly enriched. Gene Ontology (GO) analysis showed a significant representation of biological processes that include regulation of phosphatase activity as well as molecular function entities such as phosphatase activator activity. These features are consistent with the functional roles of EGFR signaling and suggest that the enriched EGFR interactors are accurately represented.

It was orthogonally confirmed that six top hits were biotinylated by biotin-IP, where streptavidin pull-down samples were analyzed by WB using specific antibodies following proximity labeling (FIG. 6D). Among them, five were observed to co-IP with EGFR (FIG. 6D). All six proteins are known to either functionally interact with EGFR or found in immunoprecipitation experiments. These include ITB1, which is critical for stable maintenance for EGFR on the cell membrane, as well as macrophage migration inhibitory factor (MIF), an immunostimulatory cytokine regulated by matrix metalloproteinase 13 (MMP13) known to be inhibitory for EGFR activation. Others include substrates of EGFR such as glutathione S-transferase P1 GSTP1 and tight junction protein ZO1, both of which are known to be activated upon phosphorylation by EGFR. One target membrane-associated progesterone receptor component 1, PGRC1, was not observed in EGFR co-IP experiment. Without intending to be bound by theory, it is believed that some interactions were not strong enough to survive the co-IP workup in these cells.

### Multi-Scale EGFR Interactome Profiled Via MultiMap

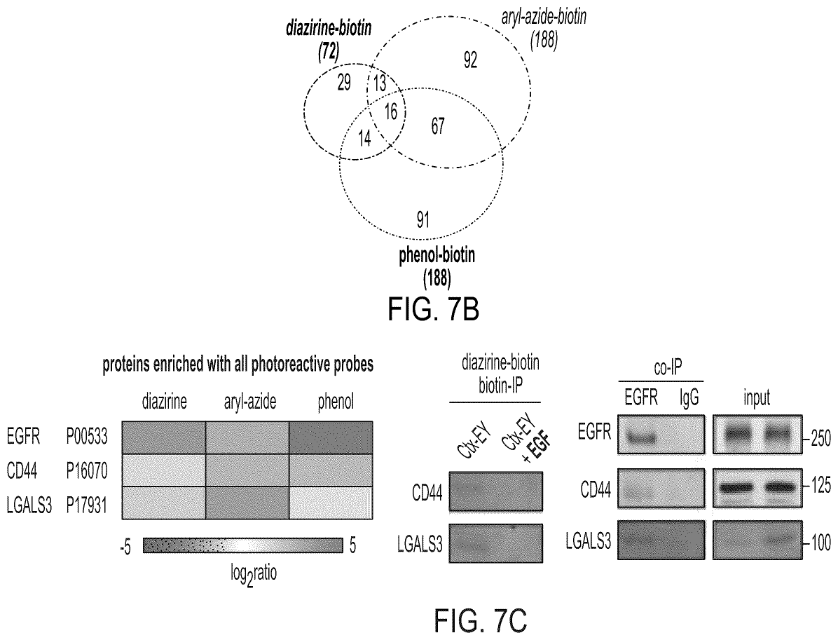

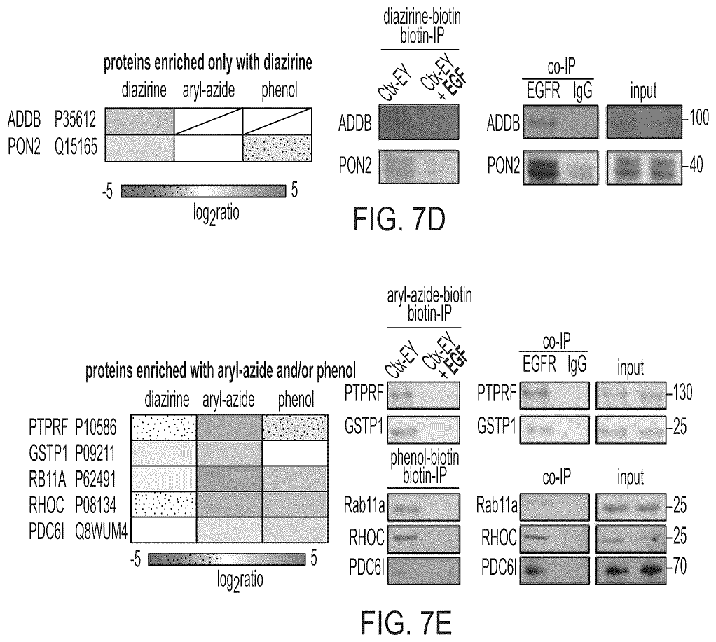

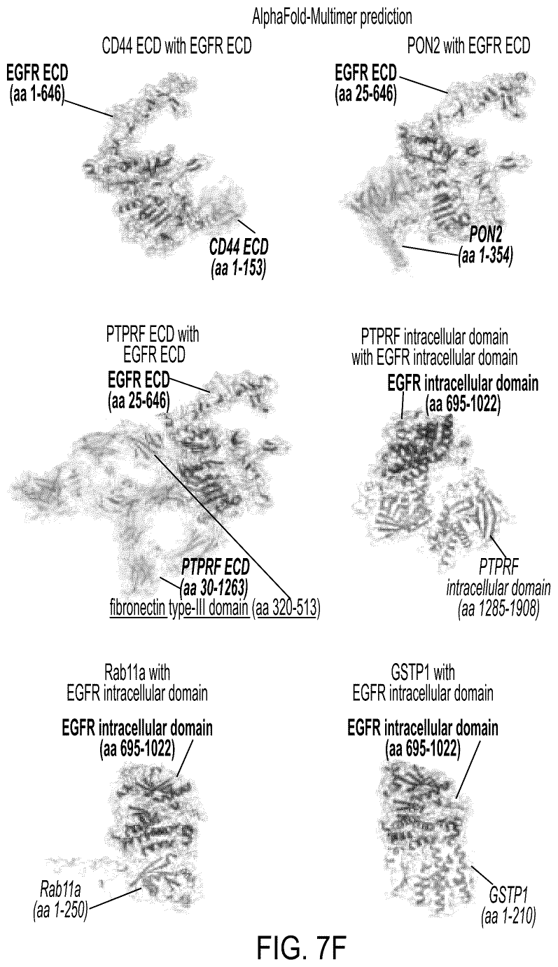

Having demonstrated the proteomic workflow of Ctx-EY triggered biotinylation on cells expressing high levels of EGFR, cells expressing modest levels of EGFR were investigated. Lung cancer cell line, A549, for example, express lower amounts of EGFR (nTPM=59.7), which is more typical of native membrane proteins. All photo-probes were applied and EGFR was selectively biotinylated with each probe. Applying the proteomics workflow, the EGFR neighbors enriched with diazirine-biotin, aryl-azide-biotin and phenol-biotin were identified by comparing labeling with Ctx-EY in the absence and presence of EGF (FIG. 7A). It was found that EGFR is one of the most enriched proteins from all three datasets. Enriched proteins were identified with the same statistical thresholds [log 2(ratio)≥1, p-value <0.05, unique peptide ≥2], allowing direct comparison of protein identities across reactions with different photo-probes. 72 proteins were identified using diazirine-biotin, 188 using aryl-azide-biotin, and 188 using phenol-biotin.

As represented in a Venn diagram (FIG. 7B), there were a total of 322 unique proteins enriched over the controls in at least one of the three photo-probes. The aryl-azide-biotin and phenol-biotin labeled more proteins than diazirine-biotin reflecting their higher yields and their relatively long labeling radii. It was found that >80% of the enriched proteins were annotated as plasma membrane proteins in UniProt (plasma membrane, GO:0005886) for all three photo-probes. GO enrichment analysis suggested molecular functions such as EGFR activity and EGF binding were highly enriched.

Sixteen candidate neighbors were identified in all three datasets of MultiMap (FIG. 7B). While no direct structural evidence has been reported for EGFR with any of these, CD44 and Galectin-3 have been functionally associated with EGFR: CD44 regulates EGFR functions in the presence of CD147 and hyaluronan; Galectin-3 regulates EGFR localization and its interactions suggested through genetic studies in pancreatic cancers. Both targets were further validated by biotin-IP and EGFR co-IP (FIG. 7C), supporting that they are proximal neighbors of EGFR.

The 29 proteins that were in common for diazirine-biotin and aryl-azide-biotin, the photo-probes with high labeling resolutions (FIG. 7D) were then considered. Among them, a paraoxonase, PON2, was found, as well as two proteins associated with RTK phosphorylation and activation: beta-adducin ADDB and MAP kinase pathway member BRAF. Both PON2 and ADDB were detected by biotin-IP and EGFR co-IP. Interestingly, BRAF, a cytosolic protein was enriched by biotin-IP but not EGFR co-IP suggesting it is close but may not be in physical contact. Between the diazirine and aryl-azide datasets, the known EGFR functional interactors such as Tid1 and ITB1 were identified and also found in the A431 cell experiment, as well as previously unreported interaction partners including CKAP4 and RAC1.

The aryl-azide-biotin and phenol-biotin experiments contributed more proteins (293 in total). Among the top hits were the tyrosine-protein phosphatase receptor, PTPRF, glutathione transferase GSTP1, small GTPase Rab11a, Rho-related GTP binding protein RHOC and ESCRT protein PDC6I (FIG. 7E and FIG. S8E). Remarkably, all were detected by biotin-IP with ten out of eleven of these proteins co-IPed with EGFR, suggesting that they form relatively stable complexes.

To provide a structural level of analysis, AlphaFold-Multimer, an exciting extension of AlphaFold developed over the last few years that uses artificial intelligence to generate plausible models of binary protein complexes, was used. This community has developed scoring metrics such a predicted DockQ score (pDockQ), where a threshold of >0.23 retrieves 51% of true-positive interacting proteins with a false-positive rate of ˜1% in large test set models. Additional criteria can be applied including buried solvent accessible surface area (BSASA)≥500 Å², predicted local distance difference test (pLDDT)>50 for the interface residues and minimum predicted alignment error (PAE)<15 Å as described previously. As a true positive example, an AlphaFold-Multimer model of the EGF: EGFR complex was derived that closely overlaid that of the known structure of EGF: EGFR (PDB: 1IVO, RMSD between 469 atom pairs is 0.924 Å).

The AlphaFold-Multimer was applied to candidate neighbors validated by biotin-IP and/or EGFR co-IP in A431 and A549 cells and generated a total of 29 models. The average pDockQ score (0.298) and BSASA (1466 Å²) for the 29 EGFR-protein pairs were both above the established criteria suggesting direct interactions. AlphaFold-Multimer was then applied to compute models of all potential heterodimeric complexes from FIG. 6C and FIG. 7A. To increase the accuracy of the models for transmembrane proteins, the ECD (aa 1-646) and intracellular domain (ICD, aa 695-1022) of EGFR were calculated separately and paired them with the corresponding ECDs or ICDs of transmembrane protein targets. As previously described, only high-confidence AlphaFold-Multimer models [average pLDDT>50, minimum predicted Alignment Error (PAE)<15 Å] were retained and further filtering was performed using the aforementioned criteria [pDockQ score ≥0.23, BSASA≥500 Å²]. The final list of validated complexes included the binary complexes of EGFR ECD with CD44 ECD (aa 1-153, pDockQ=0.375), PON2 (pDockQ=0.372) and MIF (aa 1-115, pDockQ=0.375) (FIG. 7F). In addition, the ICD of EGFR is predicted to bind Rab11a (pDockQ=0.264), GSTP1 (pDockQ=0.535) and RAC1 (pDockQ=0.387) (FIG. 7F). Most interestingly, one of the AlphaFold-Multimer complexes predicted with the highest confidence is a cell-surface phosphatase PTPRF, where PTPRF ECD binds EGFR ECD (pDockQ=0.429) and likewise, the PTPRF ICD binds the EGFR ICD (pDockQ=0.476) (FIG. 7F down).

Capture of Distal Synaptic Protein Networks with MultiMap

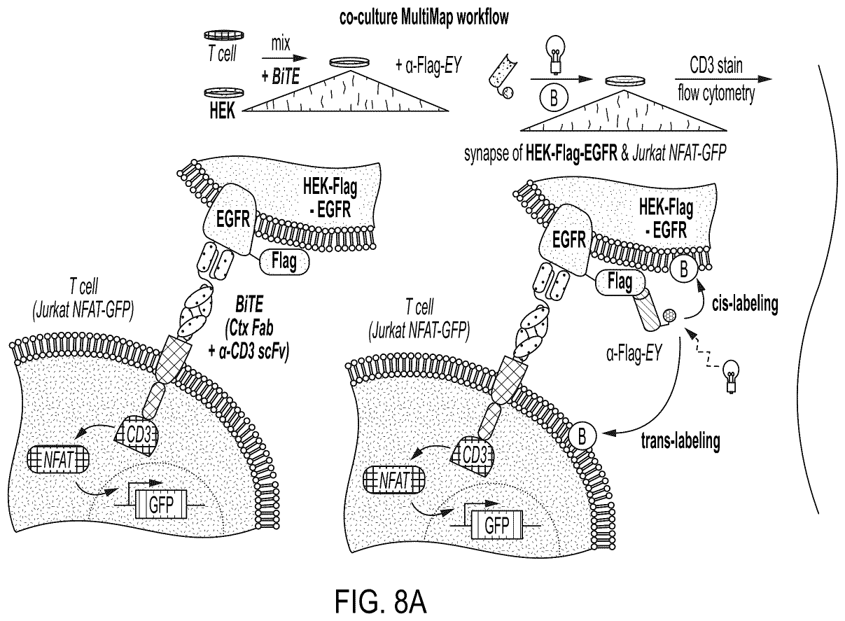

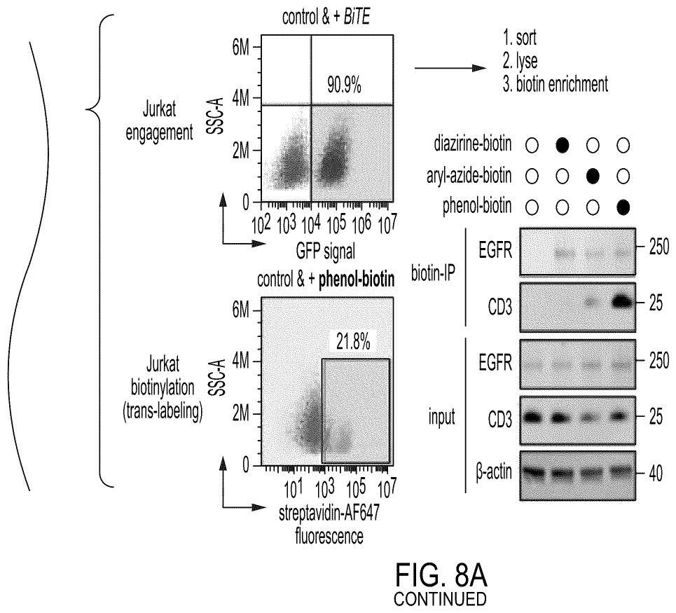

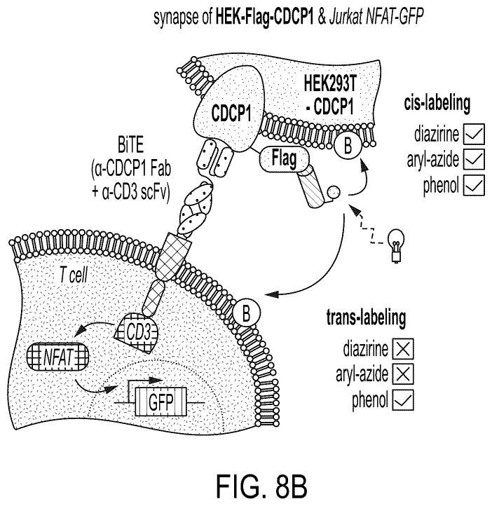

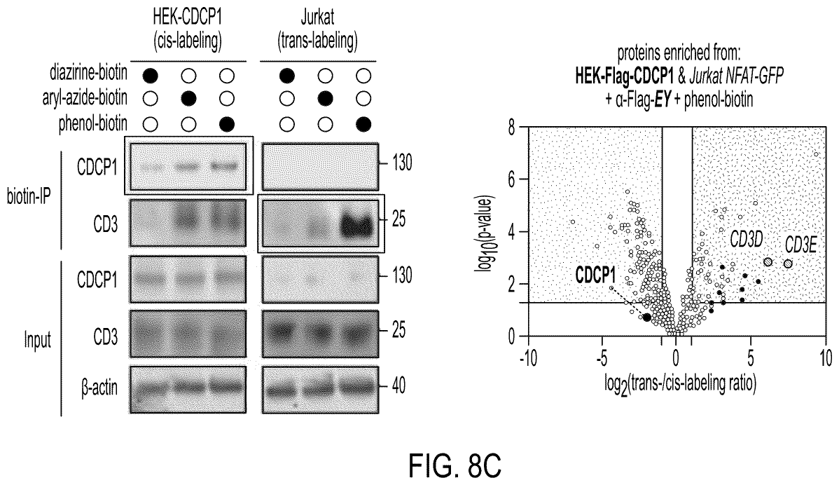

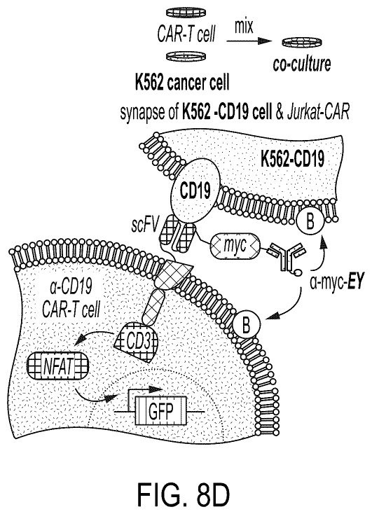

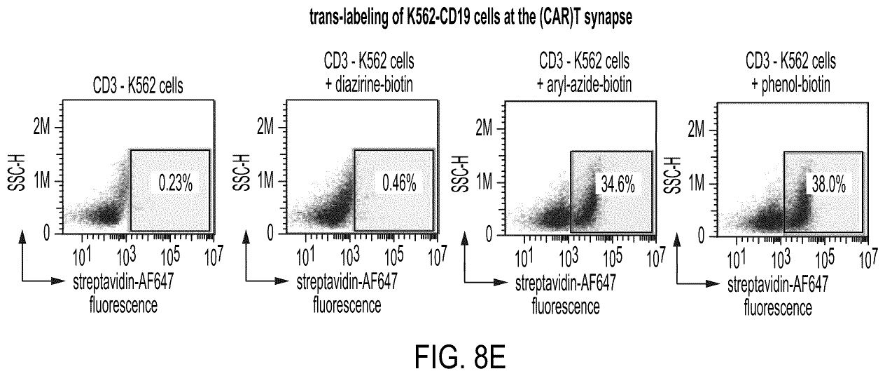

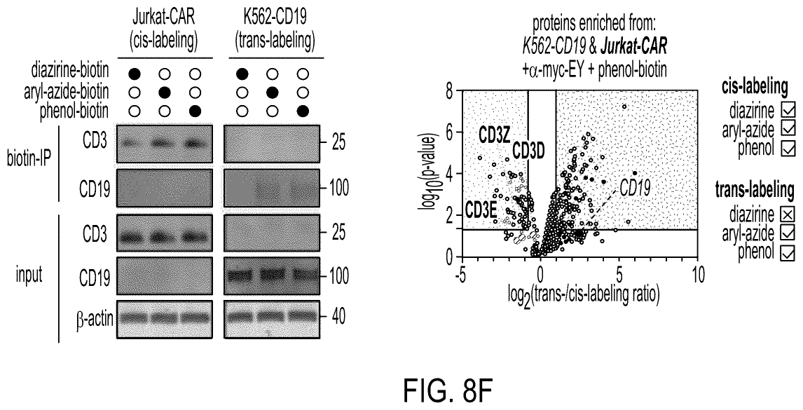

Extracellular protein-protein interactions occur not only in cis on the cell membrane but also in trans between cell-cell junctions. To explore PLP of cell synapses using MultiMap at different labeling radii, a co-culture system where the cell-cell interaction was induced by a bispecific T cell engager (BiTE) was assembled (FIG. 8A). This BiTE contained the Ctx Fab genetically fused to an ci-CD3 scFv (OKT3). Two different cells were utilized in the co-culture system: a HEK293T cell engineered to overexpress a Flag-tagged-EGFR (HEK-Flag-EGFR) and well-established Jurkat cells expressing a NFAT-GFP reporter. In this design, the Flag tag served as an orthogonal ecto-epitope for an EY-conjugated ci-Flag nanobody (ci-Flag-EY), allowing an alternative strategy of selectively recognition apart from direct antibody recognition in our EGFR studies. In order to separately characterize the labeling on HEK-Flag-EGFR and Jurkat NFAT-GFP cells, ci-CD3-PE signal was used to allow facile separation of CD3+ Jurkat cells from CD3− HEK-Flag-EGFR via FACS sorting. Levels of cis- and trans-labeling from ci-Flag-EY were determined by flow cytometry. Proteins labeled with different photo-probes were enriched using streptavidin beads and analyzed by WB (FIG. 8A).

BiTE engagement was monitored between HEK-Flag-EGFR and Jurkat NFAT-GFP cells using the standard GFP reporter readout. Dose-dependent BiTE activation of cell-cell engagement was observed, with an 80.3% shift of GFP signal in the presence of 8 nM EGFR BITE and 92.3% with 50 nM BiTE. The GFP expression was not affected by the presence of ci-Flag-EY, indicating that the Flag ecto-epitope recognition did not interfere with the cell-synapse engagement. The MultiMap workflow was then performed using four photo-probes of increasing labeling range: diazirine-biotin, aryl-azide-biotin, biocytin-hydrazide and phenol-biotin. Biotinylation was monitored in cis for HEK-Flag-EGFR and in trans for Jurkat NFAT-GFP using a streptavidin-AlexaFluor647 signal (FIG. 8A). Cis-labeling of HEK-Flag-EGFR cells occurred for >60% of cells for the diazirine-biotin, aryl-azide-biotin and phenol-biotin with ˜29% for the biocytin hydrazide. In sharp contrast, in cells overexpressing EGFR without the Flag tag, minimal shift (˜3-4%) was observed on these controls, suggesting that biotinylation induced by α-Flag-EY is highly selective. Interestingly, the trans-labeling of the Jurkat cells using the shorter-range diazirine-biotin, aryl-azide-biotin was limited to 3-4% (FIG. S10C), while the intermediate-range biocytin-hydrazide and long-range phenol-biotin labeled 9% and 22%, respectively) (FIG. 8A). This is consistent with the cell-cell synapse distance based on the length of the Fab-ScFv BiTE, plus the size of the EGFR ECD and the CD3 complex. By further analysis via WB, the cis-target EGFR was observed enriched by biotin-IP in the presence of the three photo-probes, whereas trans-target CD3 was only significantly enriched in the phenol-biotin sample, with moderate amount observed in the aryl-azide-biotin-labeled sample (FIG. 8A). Thus, longer-range photo-probes are more efficient for trans-labeling, which is consistent with previous studies.

To further expand the generality of MultiMap for cell-cell synapses, the BiTE system was tested with two other cancer targets, HER2 and CDCP1 (FIG. 8B). We fused α-HER2 Fab sequence (Trz Fab) and a previously generated α-CDCP1 Fab (4A06) onto the CD3 scFv scaffold (FIG. 8B). Again, both dose-dependent cell-cell engagement was observed in the presence of the engineered BiTEs and antigen-expressing cells as well as similar biotinylation pattern: cis-labeling was found with all three photo-probes and trans-labeling activated primarily with phenol-biotin. By introducing EY directly on the BiTE construct, confocal imaging in the HEK-Flag-CDCP1/Jurkat NFAT-GFP co-culture system was performed and it was confirmed that biotinylation primarily occurred at the cell-cell synapse.

To examine the proteins at the cell synapse, HEK293T-CDCP1 and Jurkat-NFAT-GFP cells were separated using FACS sorting and enriched biotinylated proteins from either cell. Selective biotinylation of CDCP1 with all three photo-probes, and CD3 only with phenol-biotin was confirmed (FIG. 8B). The proteins captured at either side of the cell synapse between HEK-Flag-CDCP1 and Jurkat NFAT-GFP were quantitatively profiled (FIG. 8C). It was discovered that CDCP1 was enriched in the cis-labeled samples. Proteins from the CD3 complex including CD3d and CD3e were highly enriched in the trans-labeled samples. Similarly, CDCP1 and CD3 components were also selectively enriched in BiTE-EY labeled samples when compared with an IgG isotype control. Additional CDCP1 epitope-free control using wild-type HEK293T confirmed that the selective protein enrichment is epitope-dependent. These results demonstrate that the MultiMap workflow can selectively label proteins at the BiTE-induced cell-cell synapse.

Lastly, MultiMap labeling at a (CAR)T cell-cell synapse was investigated (FIG. 8D). Jurkat cells expressing a Myc-tagged CAR construct (Jurkat-CAR) that targets CD19 were mixed with K562 cancer cells expressing CD19 ectodomain (K562-CD19). With an EY-conjugated α-myc antibody (α-myc-EY), the workflow was performed by introducing cis-labeling on K562-CD19 and trans-labeling on Jurkat-CAR cells. Cell engagement was confirmed by monitoring the CAR activation with or without K562 cells. Cis-labeling was observed upon interacting CAR cells with all photo-probes. On the other hand, both aryl-azide-biotin and phenol-biotin achieved trans-labeling, with much lower level of biotinylation using the short-range diazirine-biotin (FIG. 8E). The same results were confirmed by WB analysis (FIG. 8F).

These results are in line with the estimate of cell-cell distance between CAR-induced synapse at ˜120 Å according to AlphaFold prediction, which is shorter than BiTE-induced synapse. Each cell type was sorted for proteomics analysis and found both CD19 and CD3 component enriched for trans-labeling and cis-labeling (FIG. 8F). Direct comparison of labeled proteins at the CAR-T synapse with the isotype and CD19-epitope-free controls further confirmed the selectivity in synaptic labeling. Taken together, the data suggests that MultiMap can label cells at the cell-cell synapses and map the proteins in proximity via PLP. Only minimal alternation to the existing workflow is needed for labeling in different cell-cell engagement scenarios. This platform will be a useful technology to identify key proteins in different synaptic environments.

### General Methods and Instrumentation.

Illumination was performed using a Penn PhD Photoreactor M2 (Sigma Aldrich, Z744035, or equivalent) with a 450 nm blue light source module (Sigma Aldrich, Z744033) at 100% intensity, or LED array light sources (Thor Labs, LIU470A or equivalent for 470 nm LED array, LIU525B or equivalent for 525 nm LED array) along with a LED mounting adapter (AD38 or equivalent). To use Penn PhD Photoreactor, fan speed was set at 6800 rpm under manual control with 100/min stirring and samples were illuminated at 100% intensity for indicated time. To use LED array light sources, samples were placed under a Thor Labs LED array light source, which provides 4.0 mW/cm2 (470 nm) and 1.9 mW/cm2 (525 nm) intensity at 100 mm distance from the LED according to information from the manufacturer. Flow cytometry experiments were performed on a CytoFlex flow cytometer (Beckman CytoFlex or equivalent) and analyzed using FlowJo software. Cell sorting experiments were performed on a SONY cell sorter (SONY, SH800 or equivalent). Proteomics experiments were performed on a TimsTOF PRO (Bruker) or equivalent, equipped with a CaptiveSpray source and a nanoElute System. The peptides were separated on a 25 cm, ReproSil c18 1.5 UM 100 Å column (PepSep, PN. #PSC-25-150-15-UHP-nc). Protein quantification was performed by bicinchoninic acid assay on a multimode microplate reader Infinite 200 PRO or equivalent (Tecan Trading AG, Switzerland). Sonication of cells or protein pellets was performed using a QSonica Q500 Sonicator or equivalent (QSonica Sonicators, Newtown, CT). DNA, RNA or protein concentrations were measured using a NanoDrop 2000 spectrophotometer or equivalent (Thermo Scientific).

For immunoblotting analysis, proteins were loaded on 4-12% BisTris gels (Bolt 4-12% 17-well, Thermo Fischer, NW04127BOX), and transferred from SDS-PAGE gels to PVDF membranes (Thermo Fischer, IB24002) using an iBlot-2 dry blotting system (Thermo Scientific, IB21001). Membranes were blocked with Tris buffered saline (TBST, 37 mM sodium chloride, 20 mM Tris, 2.7 mM potassium chloride, 0.05% Tween 20; pH=7.4) containing 0.1% Tween-20 and 5% BSA and incubated with the primary antibodies and the secondary antibodies sequentially including anti-rabbit IgG Goat IR800 secondary antibody (Rockland, 926-32211), anti-rabbit IgG Goat IR680 secondary antibody (Rockland, 611-144-002), anti-rabbit IgG Goat secondary antibody peroxidase (Rockland, 611-1302), anti-mouse IgG Goat IR800 secondary antibody (Rockland, 610-145-211) and anti-mouse IgG Goat IR680 secondary antibody (Rockland, 610-144-002). Immunoblots images were captured by an infrared LI-COR imager (Odyssey CLx). In-gel fluorescence and immunoblot fluorescence signals were detected on a BioRad imager (ChemiDoc XRS+ System).

### General Chemical Methods and Instrumentation.

Chemicals were purchased including TFPA-PEG3-biotin (Thermo Scientific, 21303), biotinyl tyramide (Sigma-Aldrich, SML2135), eosin-5-isothiocyanate (Biotium, 90091), 5-iodoacetamidoerythrosin (Alfa Chemistry, ALP3853), erythrosine B disodium salt (Alfa Aesar, A14180-14), DBCO-PEG4-amine (Click Chemistry Tools, A103P-100; separately synthesized by ChemPartner), 2-(Prop-2-yn-1-yloxy)-4-(3-(trifluoromethyl)-3H-diazirin-3-yl)benzoic acid (Sigma-Aldrich, 900858), Eosin Y (Sigma-Aldrich, E4009) and Rose Bengal (Sigma-Aldrich, 33000). All solvents and reagents were purchased from chemical suppliers (Sigma Aldrich, Acros Organics, Thermo Scientific or VWR Chemicals BDH®) and were used as received unless otherwise noted. Flash Column Chromatography was performed using Teledyne ISCO CombiFlash EZ Prep chromatography system, employing pre-packed silica gel Teledyne ISCO RediSep cartridges. Protein mass spectra were obtained using a Waters Xevo G2-XS time-of-flight mass spectrometer operating with Waters MassLynx software (version 4.2). DBCO-PEG3-EY was synthesized and characterized as shown in Scheme 1 (ChemPartner). Diazirine-biotin (diazirine-PEG3-biotin) was synthesized and characterized according to published reports (Medicilon).

Proton nuclear magnetic resonance spectrum (1H NMR) and carbon nuclear magnetic resonance spectrum (13C NMR) were recorded on a Bruker 400 MHz or equivalent instrument at 25° C. Chemical shifts were reported in parts per million (ppm, 0 scale) relative to residual solvent as an internal reference (DMSO: 2.50 ppm for 1H and 39.52 ppm for 13C). Data are represented as follows: chemical shift, multiplicity (s=singlet, d=doublet, t=triplet, q=quartet, quin=quintet, m=multiplet and/or multiple resonances, br=broad, app=apparent), integration, coupling constant (J) in Hertz (Hz), and assignment. Infrared (IR) spectrum was recorded on a Bruker ALPHA FT-IR or equivalent and are reported in terms of frequency of absorption (cm-1) and intensity of absorption (s=strong, m=medium, w=weak, br=broad).

### Antibodies and Biological Reagents.

Antibodies were purchased including: Ctx (cetuximab, Selleck Chemicals, A2000), Trz (trastuzumab, Selleck Chemicals, A2007), anti-EGFR (Thermo Scientific, MA5-13319; Cell Signaling Technology, 4267S), anti-HER2 (Cell Signaling Technology, 2165S), anti-MIF (Proteintech, 20415-1-AP), anti-GSTP1 (Proteintech, 15902-1-AP), anti-ZO1 (Proteintech, 21772-1-AP), anti-PGRMC1 (Cell Signaling Technology, 13856T), anti-Integrin 131 (Cell Signaling Technology, 4706S), anti-13-actin (Santa Cruz Biotechnology, sc-47778), anti-LGALS3 (Cell Signaling Technology, 12733S), anti-CD44 (Cell Signaling Technology, 3578S), anti-ADDB (Proteintech, 14640-1-AP), anti-PON2 (Abcam, ab183710), anti-PTPRF (anti-LAR, R&D system, MAB3004-SP), anti-Rab11a (Cell Signaling Technology, 2413S), anti-RHOC (Cell Signaling Technology, 3430T), anti-PDCD6IP (Proteintech, 12422-1-AP), anti-BRAF (Cell Signaling Technology, 14814S), anti-Tid1 (Cell Signaling Technology, 4775S), anti-CKAP4 (Proteintech, 16686-1-AP), anti-Rac1/2/3 (Cell Signaling Technology, 2465T), anti-DOCK4 (Proteintech, 21861-1-AP), anti-SIGMAR1 (Proteintech, 15168-1-AP), anti-PKACa (Cell Signaling Technology, 4782S), anti-DNAJC13 (Bethyl Laboratories, A304-872A), anti-13-catenin (Cell Signaling Technology, 8480T), anti-Flot2 (anti-Flotilin 2, Cell Signaling Technology, 3436S), mouse IgG1 isotype control (BD Biosciences, 556648), anti-CD3D (Cell Signaling Technology, 31857S), anti-CD19 (Cell Signaling Technology, 90176T), anti-CDCP1 (Cell Signaling Technology, 4115S) and anti-Myc (Santa Cruz Biotechnology, sc-40). Anti-M1-FLAG antibody was purified in HEK293T cells using the sequence gifted by the Kruse lab (Harvard Medical School).

The following antibodies were used in flow cytometry assays: anti-CD3-AlexaFluor561 (Thermo Scientific, 505-0038-41), anti-CD3-PE (BioLegend, 300456), anti-CD19-PE (BioLegend, 302254), streptavidin-AlexaFluor488 (Thermo Scientific, S32354), streptavidin-AlexaFluor647 (BioLegend, 405237), anti-EGFR-AlexaFluor647 (Fisher Scientific, 352918), anti-human IgG-AlexaFluor488 (BioTechne, FAB110G) and anti-human IgG-AlexaFluor647 (BioTechne, FAB110R). Recombinant proteins included human EGFR (Bio-Techne, 1095-ER-002) and human HER2 (Acro Biosystems, HE2-H5225). Gels were imaged with InstantBlue protein stain (Expedeon, ISB1L). Albumin was purchased from Sigma-Aldrich (A1887). For enrichment assays, NeutrAvidin agarose beads (Pierce, 29200) and protein A magnetic beads (Cell Signaling Technology, 73778) were used. Cell lysis buffer was prepared by diluting from 10× cell lysis buffer (Cell Signaling Technology, 9803S) or from 10×RIPA buffer (EMD Millipore, 20-188). Sample loading buffer were diluted from 4×LDS sample loading buffer (G Biosciences, 786-323). Sequencing-grade modified trypsin (Promega, V5111), sequencing-grade chymotrypsin (Promega, V1061) and mini Bio-Spin columns (Bio-Rad, 7326207) were purchased. When performing solvent exchange processes, 7 kDa Zeba Spin desalting columns (Thermo Fischer, 89883) were used.

### Plasmid Construction.

Plasmids for the Ctx-OKT3 BiTE, Trz-OKT3 BiTE and α-CDCP1-OKT3 BiTE that targeted EGFR, HER2 and CDCP1, respectively were constructed by standard molecular biology methods and as previously described. For example, DNA fragments of Ctx Fab heavy and light chain were synthesized by integrated DNA technologies (IDT). OKT3 scFv was amplified using cloning primers. All BiTEs were constructed in the pFUSE-hIgG1 vector (InvivoGen) with IL-2 signal peptide for mammalian expression. Ctx Fab heavy chain was cloned on one vector, and the Fab light chain genetically fused with the N-terminus of OKT3 was cloned on a separate copy of the vector. The sequence of the linker between the light chain and scFv is as follows: GGGGS. All sequences were confirmed by Sanger (Quintarabio) and whole-plasmid (Primordium Labs) sequencing.

### Cloning Primers:

(forward)
5′-CCGGGGGGAATGTGGCGGCGGAGGCAGCGACATCAAGCT
GCAGCA-3′
(reverse)
5′-ATCTTATCATGTCTGGCCAGCTAGCTCACTTCAGTTCC
AGCTTTG-3′.

### Cell Culture.

A549, A431, NCI-H441, SKBR3, Jurkat and HEK293T cells were all purchased from the UCSF cell culture facility. A549, A431, NCI-H441 and SKBR3 cells were cultured and maintained in ATCC recommended conditions. HEK293T cells with Flag-CDCP1 overexpressed were generated according to literature. HEK293T cells with Flag-EGFR overexpressed were generated similarly. Jurkat cells expressing NFAT-GFP reporter were cultured in RPMI containing 10% FBS, 1% pen/strep and 2 mg/mL geneticin. K562-CD19 and Jurkat-CAR cells were cultured according to literature.

### Mammalian Protein Expression.

HEK293Expi (Expi293) cells were cultured in FreeStyle Expi293 media (Gibco, 12338018) at 37° C. and 8% humidity with orbital shaking at 250 rpm. Protein expression plasmids were cloned into a pFUSE vector (InvivoGen) with upstream IL-2 secretion signal. Cells were transfected at 3M/mL density using FectoPRO transfection kit (Genesee Scientific, 55-332) according to manufacturers' instructions. After expression for 4-6 days, the supernatant from Expi293 cells was collected by centrifuging at 4000 g for 30 min and filtered through a 0.45 μm filter. After equilibrating Hitrap Protein A/L affinity column (GE Healthcare, 12-0402-01) or nickel resin, columns were washing with PBS (pH 7.4) using six times the column volume, and protein was eluted into 100 mM acetic acid. Following pH neutralization, the purified proteins were buffer exchanged with PBS (pH 7.4) using 10 kDa MW spin filters (AmiconUltra, UFC9010). Protein samples prepared in 4× loading dye with or without DTT were then characterized using SDS-PAGE. Purified proteins were quantified using A280 channel on a NanoDrop, and flash frozen in single use aliquots for storing at −80° C. or used fresh within a week.

General Protocol for Antibody Conjugation with EY.

In order to generate antibody-EY conjugates, different bioconjugation strategies were employed including NHS labeling and oxaziridine labeling. For NHS labeling, a 200 μL reaction mixture was prepared with final concentrations of 10 μM purified antibody and 50 μM N-Hydroxysuccinimidyl-4-azidobenzoate (NHS-azide, Lumiprobe, 63720) along with 10 mM sodium bicarbonate in PBS. The reaction was incubated for 1 h at 25° C. before another portion of NHS-azide was added to reach a final concentration 100 μM. The resulting mixture was allowed to react for additional 1 h at 25° C. Then the conjugate was purified using a 7 kDa Zeba Spin desalting column (Thermo Scientific, 89882). The resulting azide-conjugated antibodies were then incubated with 100 μM DBCO-PEG4-EY for 16 h at 4° C. before purification with a 7 kDa Zeba Spin desalting column twice. Formation of the desired antibody-NHS-EY conjugates was confirmed by LC-MS, SDS-PAGE and UV-Vis spectrum scanning. The concentrations of proteins were calculated from SDS-PAGE gels. After characterization, the antibody-NHS-EY conjugate was flash frozen for future usage or used fresh within a week. For oxaziridine labeling, a 200 μL reaction was prepared with 10 μM purified antibody and 50 μM oxaziridine-azide (piperidine-oxaziridine 8 synthesized accordingly literature) in PBS. The reaction was incubated for 1 h at 25° C. before purification using a 7 kDa Zeba Spin desalting column. The resulting azide-conjugated antibodies were then incubated with 50 μM DBCO-PEG4-EY for 16 h at 4° C. before purification with a 7 kDa Zeba Spin desalting column twice. Formation of the desired antibody conjugate was confirmed as described above. After characterization, the corresponding antibody-Ox-EY conjugate was flash frozen for future usage or used fresh within a week.

### Western Blot Protocol.

Cells were incubated at 37° C. in 5% CO2 to 80% confluency and washed with 5 mL PBS three times before they were incubated with PBS with 0.04% EDTA (free of calcium and magnesium) for 15 min. Dissociated cells were collected and washed with 10 mL PBS three times before they were pelleted via centrifugation at 300 g for 5 min in 1.5 mL Eppendorf tubes. Cell pellets were resuspended in 1 mL 1×RIPA lysis buffer (EMD Millipore) supplemented with 1× cOmplete™ protease inhibitor cocktail (Roche). After 15 min incubation on ice, cells were sonicated for 15 sec (5 sec on, 5 sec off, 20%). Cell lysates were then cleared by centrifugation at 20,000 g for 10 min at 4° C. Protein concentrations were measured using a BCA assay kit (Pierce). Samples were then analyzed by SDS-PAGE and transferred onto PVDF membranes using an iBlot2 transfer stack. Total protein was first assessed using Ponceau S staining. The membranes were then blocked using TBST with 5% BSA for 1 h at 25° C. before primary and secondary antibodies were added. Biotinylation and near-infrared Western blot imaging were conducted using an Odyssey Li-COR imaging system before further analysis using ImageStudioLite.

### General Flow Cytometry

Cultured cells or co-culture systems were incubated at 37° C. in 5% CO₂ for the duration of the assay. Cells were first washed three times with 5 mL PBS followed by additional three washes with 5 mL filtered 3% BSA in PBS. Then the cells were either directly resuspended in 0.5 mL PBS for flow cytometry analysis or stained with corresponding primary antibody for 1 h at 4° C. The stained cells were then washed three times with 5 mL filtered 3% BSA in PBS before they were resuspended in 0.5 mL PBS for flow cytometry analysis. Flow cytometry data were analyzed on FlowJo.

### On-Cell Antibody Binding and Biotinylation Assay

A431, A549 or NCI-H441 cells were incubated at 37° C. in 5% CO₂ to 80% confluency and washed with PBS three times before they were incubated with PBS with 0.04% EDTA (free of calcium, magnesium) for 15 min. Dissociated cells were collected and washed three times with 10 mL PBS before they were pelleted in 1.5 mL Eppendorf tubes. Cells were resuspended in pre-chilled PBS to 1×10⁶ cell/mL concentration, and then incubated with or without antibody or reagents as indicated at 4° C. Mixtures were illuminated with LED at 4° C., pelleted and washed again three times with 0.5 mL PBS. Treated cells were then stained with streptavidin-AlexaFluor488 (1:2000 diluted with filtered 3% BSA in PBS) and/or α-human IgG-AlexaFluor647 (1:2000 diluted with filtered 3% BSA in PBS) for 1 h at 4° C. before they were washed three times with 0.5 mL filtered 3% BSA in PBS. Samples were then suspended in 0.5 mL PBS before flow cytometry analysis.

### Recombinant Protein Biotinylation Assay

In order to test the catalytic function of EY when conjugated on an antibody, both self-labeling and target-biotinylation were validated using recombinant proteins. A 100 μL reaction system in PBS was prepared with 10 μM purified antibody or antibody-EY conjugate with or without equivalent amount of binding antigen for 15 min at 4° C. For example, 10 μM Ctx or Ctx-EY conjugate was incubated with or without 10 μM recombinant EGFR in PBS. Photo-probe (diazirine-biotin, aryl-azide-biotin, biocytin-hydrazide, or biotin-phenol) was then added into the solution to reach a final concentration of 100 μM and mixed thoroughly before illumination with LED for 10 min at 4° C. Afterwards, proteins were precipitated with pre-chilled acetone to get rid of excess small molecules, resuspended in PBS or sample loading buffer, and subjected to SDS-PAGE or LC-MS/MS sample preparation. Photo-probe modifications were searched as a dynamic modification with the following mass shift: diazirine-biotin (+616.25 Da), aryl-azide-biotin (+620.23 Da), phenol-biotin (+361.15 Da) or biocytin-hydrazide (+384.50 Da).

### General Protocol for Antibody-EY Labeling on Cells

A431, A549 or NCI-H441 cells were incubated at 37° C. in 5% CO₂ to 80% confluency and washed three times with PBS before they were incubated with PBS with 0.04% EDTA (free of calcium, magnesium) to dissociate. Dissociated cells were collected and washed with 5 mL PBS three times before they were pelleted in 1.5 mL Eppendorf tubes and resuspended in pre-chilled PBS to 10M cell/mL concentration. Indicated amounts of antibody-NHS-EY conjugates were pre-chilled and added to the cells for 15 min at 4° C. before excessive antibody-EY conjugates were removed by washing with 1 mL pre-chilled PBS. The antibody-bound cells were then resuspended in 1 mL pre-chilled PBS. Photo-probe (diazirine-biotin, aryl-azide-biotin, biocytin-hydrazide, or biotin-phenol) was then added into the cell solution to reach a final concentration of 100 μM and mixed thoroughly before illumination with LED for 10 min at 4° C. Afterwards, cells were pelleted again and subjected to flow cytometry or LC-MS/MS sample preparation.

### Sample Preparation for LC-MS/MS Analysis.

For sample processing, cell pellets were resuspended in 1 mL 1×RIPA lysis buffer (EMD Millipore) supplemented with 1× cOmplete™ protease inhibitor cocktail (Roche). After 15 min incubation on ice, cells were sonicated for 15 sec (5 sec on, 5 sec off, 20%). Cell lysates were then cleared by centrifugation at 20,000 g for 10 min at 4° C. Protein concentrations in the cleared supernatant were measured using a BCA assay kit (Pierce). Proteins were then added to 200 μL NeutrAvidin agarose beads (Pierce) that were pre-washed with 5 mL PBS for 3 times and incubated for 16 h at 4° C. Afterwards, supernatant was discarded using mini Bio-spin columns (Bio-Rad) and the beads were washed three times with 3 mL 1×RIPA lysis buffer, three times with 3 mL 1M NaCl in 1×PBS, and three time with 3 mL of freshly prepared 2M urea in 50 mM ammonium bicarbonate. The beads were then suspended in 100 μL PBS to re-constitute 50% slurry with 10 μL bead slurry separated for Western blotting.

Proteins on the washed beads were then digested using the Preomics IST kit in an on-bead digestion format according to the manufacturer's instructions. In brief, washed beads were suspended in 100 μL LYSE buffer provided by Preomics IST kit and incubated at 55° C. for 10 min for reduction and alkylation. Once the beads cooled down to room temperature, 50 μL of pre-reconstituted DIGEST were added to the beads and incubated at 37° C. for 3 h with shaking. The digested peptides were then collected using mini Bio-Spin columns (Bio-Rad) and another 50 μL of LYSE buffer were added to wash the beads. Afterwards, 100 μL of STOP solution was added to the combined flow-through elution and mixed using vigorous vortexing. Then the peptides were desalted using the Preomics desalting columns before they were dried under vacuum and resuspended in 15 μL solvent A (0.1% formic acid with 2% acetonitrile) for mass spectrometry analysis. Peptide amount was monitored by quantitative fluorometric peptide assay (Pierce).

### Proteomics Analysis of Digested Peptide Samples.

Proteomics experiments were performed on a TimsTOF PRO (Bruker) equipped with a CaptiveSpray source and a nanoElute system. The peptides were separated on a 25 cm, ReproSil c18 1.5 μM 100 Å column (PepSep, PN. #PSC-25-150-15-UHP-nc) using a step-wise linear gradient method with water in 0.1% formic acid (solvent A) and acetonitrile with 0.1% formic acid (solvent B): 5-30% solvent B for 90 min at 0.5 μl/min, 30-35% solvent B for 10 min at 0.6 μl/min, 35-95% solvent B for 4 min at 0.5 μl/min, 95% hold for 4 min at 0.5 μl/min). Acquired data was collected in a data-dependent acquisition mode with ion mobility activated in PASEF mode. MS and MS/MS spectra were collected with m/z ranging from 100 to 1700 in positive mode.

### Analysis of Proteomics Dataset.

All acquired data was searched using PEAKS online Xpro 1.6 (Bioinformatics Solutions Inc.) or FragPipe powered by MSFragger (v3.7). Spectral searches were performed using a curated FASTA-formatted dataset containing Swiss Uniprot-reviewed human proteome file with gene ontology localized the plasma membrane (downloaded from UniProt database). A precursor mass error tolerance was set to 20 ppm and a fragment mass error tolerance was set at 0.03 ppm. Peptides, ranging from 6 to 45 amino acids in length, were searched in semi-specific trypsin digest mode with a maximum of three missed cleavages. Carbamidomethylation (+57.0214 Da) on cysteines was set as a static modification while methionine oxidation (+15.9949 Da) and lysine acetylation (+42.0115 Da) were set as a variable modification. Peptides were filtered based on a false discovery rate (FDR) of 1%. Samples were normalized using total ion current (TIC). For p-value and fold change calculations, the data were further processed using a customized script, as previously described. To analyze the portions of previously identified plasma membrane or cell surface proteins among the identified hits, annotated datasets were exported from UniProt or downloaded from previous reports.

### Immunoprecipitation Assays (Co-IP) in Live Cells

For endogenous protein immunoprecipitation using protein A/G beads, cell lysates with equal amounts of protein were diluted with PBS and incubated with protein A/G beads (pre-washed three times with binding buffer, 50 mM Tris, 150 mM NaCl, 0.2% Triton, pH=7.5) for 2 h at 4° C. along with the protein-specific antibody at the vendor-suggested dilution. The beads were washed three times with binding buffer (50 mM Tris, 150 mM NaCl, 0.2% Triton, pH=7.5). The enriched proteins were eluted with acidic elution buffer (100 mM glycine, 0.1% Triton, pH 2.8) before neutralizing with 1M Tris (pH 8), according to the manufacturer's instructions.

### AlphaFold-Multimer Prediction and Analysis

In silico screening using AlphaFold-Multimer program on the ColabFold platform as previously described. In brief, AlphaFold-Multimer calculations were performed using AlphaFold-Multimer v3 on ColabFold v1.5.2 using NVIDIA A100 GPUs with sequence alignment generated through MMseqs2 and HHsearch. Predictions were generated in a combination of the paired and unpaired multiple sequence alignment, 20 recycles to generate 5 independent unrelaxed models. Sequences were obtained directly from UniProt database. All ranks are examined by both prediction confidence and accuracy. Models with an average predicted local distance difference threshold (pLDDT)>50 and minimum predicted alignment error (PAE)<15 Å were considered.

Predicted AlphaFold-Multimer binary complexes were further scored using predicted DockQ score (pDockQ) and buried solvent accessible surface area (BSASA). pDockQ scores were generated to indicate the interface accuracy quantitatively (0 is the worst and 1 is the best) with ≥0.23 cutoff value for direct binary contact as previously described. BSASA is defined as ΔSASA_{AB}=SASA_{A}+SASA_{B}−SASA_{AB}, where a 1.4 Å radii rolling probe was used to calculate solvent accessible surface area for all non-hydrogen, non-monoatomic ion atoms in chains A and B.

### Preparation of Cell-Cell Synapses Using Co-Cultured Cells

In order to activate cell-cell recognition, a mixture of two cells were prepared and counted. In the case of BiTE systems, target cell line (HEK293T-Flag-EGFR, HEK293T-Flag-CDCP1, SKBR3) were first plated and let attach to the plate for 8 h. Jurkat NFAT-GFP was then added at a 2.5:1 effector: target ratio. Indicated concentration of BiTE or BiTE-EY construct was added and incubated for more than 20 h before the cells were harvested for flow cytometry or on-cell biotinylation experiments. In the case of the CAR system, Jurkat-CAR and K562-CD19 were plated at a 2.5:1 effector: target ratio and incubated for 20 h before the cells were harvested for flow cytometry or on-cell biotinylation experiments.

### Cell-Cell Biotinylation Sample Preparation.

For intercellular biotinylation experiments, co-cultured cells were carefully washed with pre-chilled PBS. Indicated amounts of pre-chilled antibody-EY were added to the cells for 15 min at 4° C. Photo-probe (diazirine-biotin, aryl-azide-biotin, biocytin-hydrazide, or biotin-phenol) was then added into the samples and mixed thoroughly before LED illumination for 10 min at 4° C. Afterwards, cells were pelleted and wash three times with PBS. The cells were either lysed directly for LC-MS/MS sample preparation as described above or FACS sorted before sample preparation. To perform FACS sorting, cells were stained with α-CD3-AlexaFluor561 before they were sorted into CD3+ and CD3− cells using a Sony cell sorter (SH800S).

### Cell-Cell Biotinylation Confocal Imaging

Cell-cell synapses were prepared and biotinylated on an iBidi plate precoated with ibiTreat (μ-Dish 35 mm, iBidi 81156). Treated cells were gently washed twice with PBS before freshly diluted 4% paraformaldehyde was added dropwise. Cells were fixed for 10 min at 25° C. and gently washed twice with PBS. Freshly filtered 3% BSA in PBS was then added dropwise to block for 8 h at 4° C. Cells were stained with streptavidin-AlexaFluor647 (dilution 1:1000 diluted with filtered 3% BSA in PBS) for 1 h at 4° C. in dark, gently washed twice with PBS and stained with freshly diluted Hoechst DNA dye for 10 min at 25° C. in dark. The samples were again gently washed twice with PBS and imaged with an inverted Nikon Ti-E microscope equipped with a Yokogawa CSU-22 confocal scanner unit.

### Software.

Data were analyzed and visualized using Microsoft Excel (v16.22) and GraphPad Prism (v8.0.1), in addition to software listed by each experiment. NMR data were analyzed using MestReNova (v15.0.0). DNA and protein sequences were analyzed using Geneious (v10.0.7). Flow cytometry data were analyzed by FlowJo (v10.6.1) and FACS data were processed by Software Wizards installed on SH800S cell sorter. Proteomics data were analyzed by PEAKS online (Xpro 1.6) and FragPipe powered by MSFragger (v3.7). Images were made using ImageStudioLite (v5.2.5), Adobe Illustrator (v22.1) and BioRender (v2.0). Structural assignments to site-specific modifications and structural comparison were performed using ChimeraX (v1.6.1). AlphaFold-Multimer prediction was performed on ColabFold (v1.5.2) and visualized using ChimeraX (v1.6.1). Code for BSASA calculation is publicly available on GitHub and Dryad: https://github.com/ajipalar/guide_bsasa (commit 7541e6f3ba89a0089b7f01c8792a2f356264cd68).

### Statistical Analysis.

Statistical analyses (unpaired Student's t-tests) were performed using GraphPad Prism. Data were derived from at least three biological replicate experiments and presented as the mean±s.d., P≤0.05, **P≤0.01, ***P≤0.001, ****P≤0.0001 and n.s., not significant.

### Preparation of DBCO-PEG₄-EY (6)

DBCO-PEG₄-EY (6) was prepared according to the following scheme:

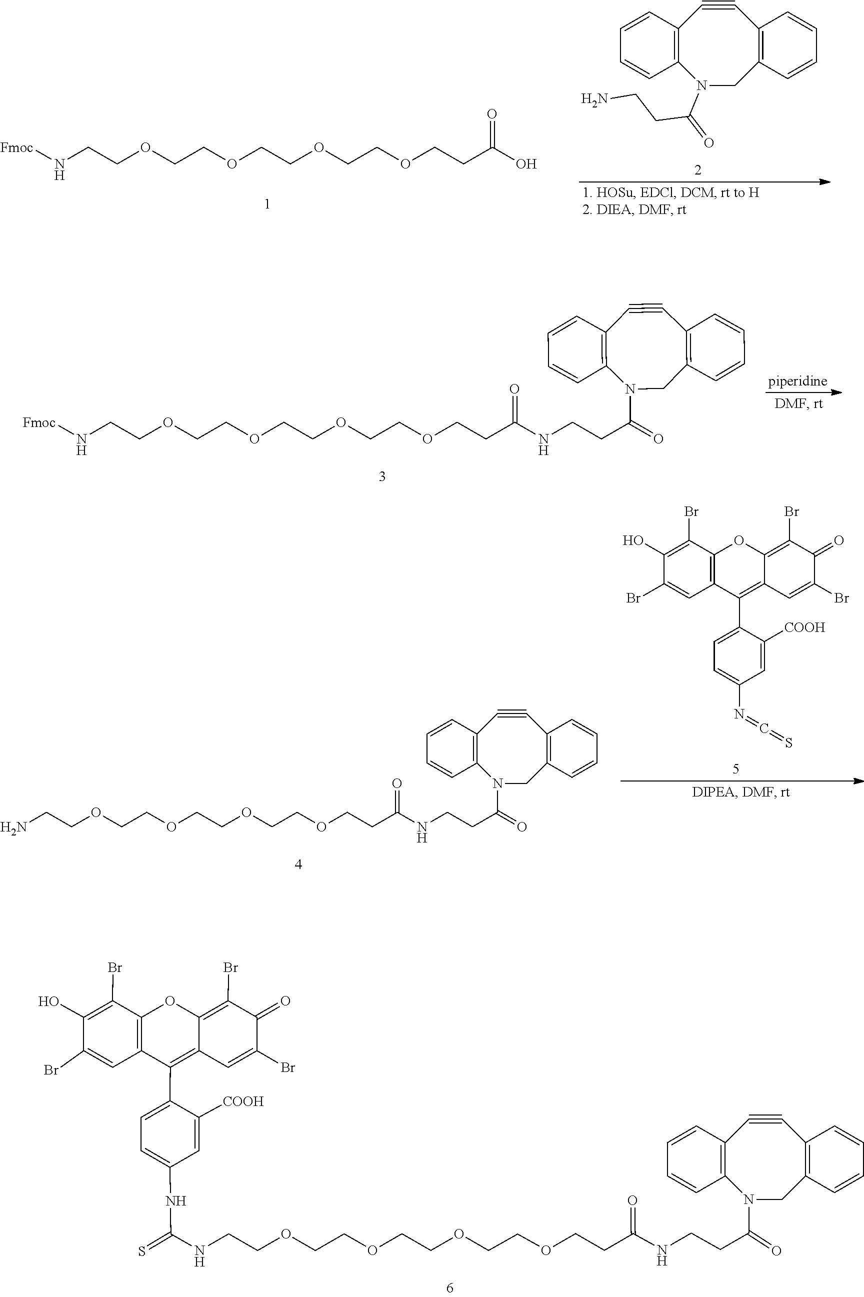

To a solution of 1-(9H-fluoren-9-yl)-3-oxo-2,7, 10, 13, 16-pentaoxa-4-azanonadecan-19-oic acid 1 (974 mg, 2 mmol, 1.0 equiv.) in DCM (20 mL) was added HOSu (460 mg, 4 mmol, 2.0 equiv.) and EDCl (764 mg, 4 mmol, 2.0 equiv.) under N₂ atmosphere. The mixture was stirred at 25° C. for 2 h. The mixture was poured into saturated sodium chloride aqueous solution (26.2 wt %) and extracted with EtOAc. The organic phase was dried over anhydrous magnesium sulfate, filtered, and concentrated in vacuum to afford a residue, which was dissolved in DMF (5 mL). DIPEA (516 mg, 4 mmol, 2.0 equiv.) was then added to the resulting solution, followed by compound 2 (552 mg, 2 mmol, 1.0 equiv.). The mixture was stirred at 25° C. for 2 h. The crude reaction mixture was purified by reverse phase HPLC (eluting with 0-65% acetonitrile in water with 0.01% TFA) to give compound 3 as a yellow solid (650 mg, 0.87 mmol, yield: 43%). m/z=746.1 [M+H]⁺.

To a solution of 3 (650 mg, 0.87 mmol, 1.0 equiv.) in DMF (5 mL) was added piperidine (148 mg, 1.75 mmol, 2.0 equiv.) under N₂ atmosphere. The reaction mixture was stirred at 25° C. for 2 h and directly purified by reverse phase HPLC (eluting with 0-65% acetonitrile in water with 0.01% TFA) to give 4 as a yellow solid (400 mg, 0.76 mmol, yield: 88%). ESI m/z=524.2 [M+H]⁺.

To a solution of 4 (300 mg, 0.56 mmol, 1.0 equiv.) in DMF (3 mL) was added DIPEA (145 mg, 1.12 mmol, 2.0 equiv.) and 5 (400 mg, 0.56 mmol, 1.0 equiv.). The mixture was stirred at rt for 2 h and directly purified by reverse phase HPLC (eluting with 0-65% acetonitrile in water with 0.01% NH4HCO3) to give 6 as a red solid (200 mg, 0.16 mmol, yield: 30%). Characterization was performed by ChemPartner. m/z=615.0 [M/2+H]⁺. ¹H NMR (400 MHz, DMSO-d6) δ 10.08 (s, 1H), 8.25 (s, 1H), 8.18 (s, 1H), 7.88 (d, J=8.2 Hz, 1H), 7.71 (t, J=5.7 Hz, 1H), 7.66-7.54 (m, 2H), 7.51-7.24 (m, 6H), 7.04 (s, 2H), 5.03 (d, J=14.0 Hz, 1H), 3.74-3.36 (m, 20H), 3.16-3.04 (m, 1H), 2.99-2.86 (m, 1H), 2.43 (ddd, J=15.3, 8.5, 6.3 Hz, 1H), 2.16 (t, J=6.5 Hz, 2H), 1.80 (ddd, J=16.0, 8.5, 5.8 Hz, 1H). ¹³C NMR (101 MHz, DMSO-d6) δ 180.43, 170.18, 169.96, 168.32, 167.08, 152.97, 151.39, 148.38, 140.88, 132.41, 130.34, 129.57, 128.99, 128.25, 128.08, 127.75, 126.84, 125.25, 123.53, 122.50, 121.44, 118.29, 114.30, 109.71, 108.10, 99.33, 69.81, 69.79, 69.69, 69.65, 69.53, 69.48, 68.44, 66.70, 54,86, 43.70, 40.15, 39.94, 39.73, 39.52, 39.31, 39.11, 38.89, 35.98, 34.99, 34.19.

### Preparation of Diazirine-PEG₃-Biotin (9)

Diazirine-PEG₃-biotin (9) was prepared according to the following scheme:

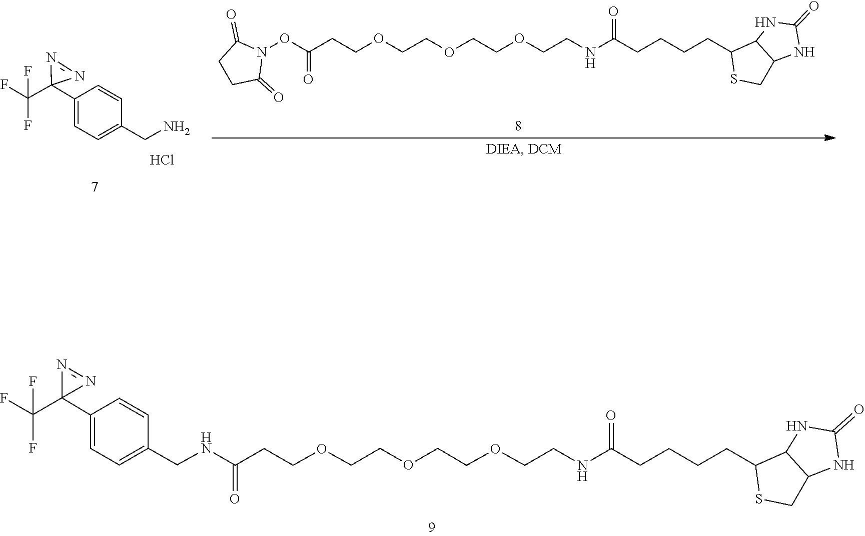 [SMILES: NCc1ccc(C2(C(F)(F)F)N=N2)cc1 ; O=C(CCCCC1SCC2NC(=O)NC21)NCCOCCOCCOCCC(=O)ON1C(=O)CCC1=O ; Cl ; O=C(CCCCC1SCC2NC(=O)NC21)NCCOCCOCCOCCC(=O)NCc1ccc(C2(C(F)(F)F)N=N2)cc1]

To a solution of [4-[3-(trifluoromethyl)diazirin-3-yl]phenyl]methanamine, hydrochloride (20 mg, 0.08 mmol, compound 7) and biotin-PEG3-NHS ester (47.7 mg, 0.088 mmol, compound 8) in dichloromethane (1 mL) was added ethylbis(propan-2-yl)amine (31 mg, 0.24 mmol). The reaction mixture was stirred for 1 h at 25° C. The reaction mixture was concentrated under reduced pressure. The residue was purified by C18 column chromatography eluted with acetonitrile:water (with 0.1% trifluoroacetic acid) (0˜40%) to afford diazirine-PEG3-biotin (7.64 mg, 13% yield, compound 9) as a white solid. Characterization was performed by Medicilon. m/z=645.2 [M+H]⁺. 1H NMR (400 MHz, DMSO-d6) δ 8.42 (t, J=6.0 Hz, 1H), 7.82 (t, J=5.6 Hz, 1H), 7.38 (d, J=8.3 Hz, 2H), 7.24 (d, J=8.0 Hz, 2H), 6.41 (s, 1H), 6.35 (s, 1H), 4.31 (t, J=5.0 Hz, 3H), 4.16-4.08 (m, 1H), 3.63 (t, J=6.3 Hz, 2H), 3.49 (s, 8H), 3.39 (t, J=5.9 Hz, 2H), 3.18 (q, J=5.9 Hz, 2H), 3.09 (ddd, J=8.6, 6.1, 4.4 Hz, 1H), 2.81 (dd, J=12.4, 5.1 Hz, 1H), 2.57 (d, J=12.4 Hz, 1H), 2.38 (t, J=6.3 Hz, 2H), 2.06 (t, J=7.4 Hz, 2H), 1.61 (ddt, J=12.2, 9.4, 6.2 Hz, 1H), 1.54-1.38 (m, 3H), 1.31 (dq, J=15.0, 7.4 Hz, 2H). ¹³C NMR (101 MHz, DMSO-d6) δ 172.21, 170.41, 162.77, 142.25, 128.05, 126.45, 125.89, 123.33, 120.60, 69.75, 69.19, 66.85, 61.08, 59.24, 55.46, 41.58, 40.15, 39.94, 39.87, 39.73, 39.52, 39.31, 39.10, 38.89, 38.47, 36.16, 35.13, 28.26, 28.23, 28.06, 27.86, 25.29. HRMS-ESI (m/z): [M+H]⁺ calculated for C28H40F3N6O6S: 645.2682. found 645.2672. IR: (ATR-FTIR) R (cm⁻¹): νmax 3289, 3087, 2927, 2871, 1702, 1640, 1551, 1462, 1423, 1345, 1232, 1184, 1143, 1052, 939, 857, 806, 725.

### Genetically Encoded Tags for Selective Live Cell Labeling

To label EGFR on either side of the membrane with the EY photocatalyst a Flag-tag was fused on the N-terminus of the extracellular domain (ECD) of EGFR and a HaloTag was fused on the C-terminus of the intracellular domain (ICD), designated Flag-EGFR and EGFR-HaloTag (Flag-EGFR-HaloTag), respectively. The Flag-EGFR-HaloTag was introduced into A549 cells, a non-small cell lung cancer cell line, for its well-established and robust response to EGF activation as well as its physiologically relevant expression level of EGFR. After transfecting the two constructs in A549 cells, significant expression of the anti-Flag signal on cells for both constructs was observed by flow cytometry. Transfection conditions were adjusted so that both engineered EGFR constructs, Flag-EGFR and EGFR-HaloTag, were at comparable levels to the endogenous EGFR.

To validate that the tagged EGFR constructs functioned properly upon EGF ligand activation, the engineered cells were incubated with EGF and EGF-stimulated cellular behavior was monitored at 5 min, 10 min, 30 min and 60 min activation in comparison with ligand-free conditions. Significant activation of well-known and functional phosphorylation sites on EGFR, Tyr1086, Tyr1045, Tyr1068, was observed at levels comparable to the wild-type EGFR. It was also confirmed that cell engineering did not significantly alter the activation patterns of downstream signaling, including ERK phosphorylation, Akt phosphorylation, SHC phosphorylation, STAT5 phosphorylation and PLCγ phosphorylation upon EGF activation. In addition, these dynamic changes were continuous throughout the 5 min, 10 min, 30 min, 60 min intervals. An EGF on-cell binding assay was performed and similar binding profiles were observed, comparing the engineered EGFR and endogenous EGFR.

The Flag-tag and HaloTag constructs were then tested for target-specific PLP. For the Flag-tag strategy, A549 cells were transfected with the Flag-EGFR construct and the cells were serum-starved for >12 hours to remove serum-induced EGF signaling. An EY-conjugated Flag antibody was added to bind to the ECD displayed Flag-tag, followed by incubation with the three photo-probes for 15 min, namely aryl-diazirine-biotin, aryl-azide-biotin and phenol-biotin, to allow cell penetrance. Cells were then illuminated with blue light to activate labeling. Significant labeling with 2 min blue light activation was observed on purified bovine serum albumin (BSA) and on live A549 cells. It was also confirmed that the labeling reactions with the photo-probes did not continue after 2 min light activation when the samples were placed in the dark, suggesting that this reaction is light-dependent, and that the temporal resolution can be controlled by external light. By performing the full workflow on cells, significant total cell labeling by flow cytometry using aryl-diazirine-biotin, aryl-azide-biotin and phenol-biotin with 2 min blue light activation was confirmed. Stronger labeling signals were observed with the aryl-azide-biotin and phenol-biotin in comparison to aryl-diazirine-biotin, which is consistent with cell surface biotinylation. Cells were lysed and biotinylated proteins were purified on streptavidin beads, trypsinized and analyzed by mass spectrometry (MS). EGFR was one of the most enriched proteins with all three photo-probes. When performing cellular localization analysis against established annotations, a significant enrichment of both plasma membrane (PM) or cell surface proteins (CSP) was observed.

To anchor EY to the ICD of the engineered EGFR-HaloTag, a EY hexyl-chloride derivative was synthesized (EY-HaloTag Ligand (EY-HTL)). Conjugation using the HaloTag and HaloTag Ligand pair is highly efficient, selective, and compatible with cellular experiments. Similar to the Flag-tag ecto-tag MultiMap workflow, the A549 cells expressing EGFR-HaloTag were incubated with EY-HTL to allow selective and covalent anchoring of the EGFR-HaloTag. To remove free EY-HTL from cells, the cells were washed four times with fresh media including 15 min soaks between washes. Flow cytometry showed dramatically increased fluorescent signal for the A549 cells expressing EGFR-HaloTag with 88% population shift compared to A549 parental cells, thus validating the washout procedure.

To test the specificity of the EY-HTL on a non-interacting target using this procedure, GFP was transfected into A549 cells with and without HaloTag and incubated with EY-HTL. Labeling experiments were conducted followed by proteomics analysis and highly selective enrichment of GFP over any other native proteins with all three photo-probes was observed. The same workflow was then performed using EY-HTL on cells expressing EGFR-HaloTag and saw significant total cell labeling with all three photo-probes. In particular, while a similar degree of labeling with aryl-diazirine-biotin and aryl-azide-biotin was observed, a lower level of overall labeling with phenol-biotin was observed; it was believed that the intracellular labeling environment may quench the phenol-biotin reaction. Thus, a panel of quenching metabolite and enzymes commonly found in cells was collected, including SOD1, glutathione, glutathione peroxidase (GPx) and catalase. Labeling with phenol-biotin on BSA and whole cell lysate with different concentrations of these additives was performed and it was found that the addition of glutathione significantly decreased phenol-biotin-based labeling in a dose-dependent manner, which supported that the intracellular component quenches the labeling efficiency. Taken together, these results confirmed that target-specific cellular labeling was achievable with genetically encoded ECD Flag-tag and ICD HaloTag and that both were compatible with the PLP workflow.

EGFR Neighborhood Changes Upon 5 Min Treatment with EGF

Signaling through EGFR is known to be transient with an activation stage followed by a deactivation and receptor turnover phase. The changes of EGFR neighborhoods upon ligand activation was monitored in a time-resolved manner. EGF-triggered EGFR processes such as phosphorylation, dephosphorylation and internalization occur rapidly within minutes to over an hour. Short light exposure (2 min) triggered ample protein labeling, thus making it possible to capture neighborhood changes with high temporal precision.

Confocal imaging showed that Flag-EGFR and EGFR-HaloTag traffic with similar kinetics and localization as the endogenous EGFR before and after EGF activation. All EGFR species (Flag-EGFR, EGFR-HaloTag and endogenous EGFR) were largely internalized within 60 min of EGF stimulation, with an overall Pearson correlation coefficient above 0.6. Time-resolved ECD ecto-tag MultiMap workflow was performed on cells in the presence of EGF. Through Western blot analysis, significant labeling on cells after EGF treatment for 5 min and 60 min, respectively, was observed.

Large-scale proteomics experiments were then conducted with ECD ecto-tag MultiMap at different timepoints by capturing biotinylated proteins on streptavidin beads, followed by trypsinization and MS analysis. It was found that a large number of proteins enriched upon 5 min EGF activation over the ligand-free state. After normalizing protein enrichment ratio to EGFR, 158 proteins increased in labeling with aryl-diazirine-biotin, 149 proteins with aryl-azide-biotin and 54 proteins with phenol-biotin were identified using the same statistical threshold [log 2(fold enrichment)≥1, P<0.05, at least 2 unique peptides, three biological replicates]. The cellular localization profiles were similar between probes, with more than 80% of the identified proteins classified as plasma membrane proteins (PM) or cell surface proteins (CSP). A significant fraction of proteins that are differential between the photo-probes were also observed. Many factors including different labeling efficiency (aryl-azide>phenol>aryl-diazirine), breadth of amino acid preference (aryl-diazirine>aryl-azide>phenol) and labeling radius driven by reactive half-life (phenol>aryl-azide>aryl-diazirine) along with detection thresholds all contribute to the differences, suggesting that all probes are useful for a more holistic view of the neighborhood.

262 enriched proteins in total were identified with many top proteins of interest and the top proteins of interest were annotated across volcano plots using different photo-probes. A number of neighbor candidates that directly interact with EGFR or functionally associate with EGFR were identified. For example, the Ras-related protein, Rab11a, is reported to co-localize with EGFR upon EGF stimulation and Rab11a overexpression accelerates EGFR recycling in cells. The EGFR pathway substrate, EPS15, was consistently found in our data and is reported to be associated with EGFR internalization and degradation. ARF6, an ADP-ribosylation factor, was found to be highly enriched upon treatment with EGF. ARF6 is reported to interact with EGFR and is dependent on EGFR palmitoylation and ARF6 myristylation. Dynamin1 (DNM1) was highly enriched and is known to regulate EGFR internalization as demonstrated in knock-out (KO) experiments. Other enriched targets with one or more photo-probes include proteins with known functional association such as the tyrosine kinase SRC⁴ DnaJ homolog subfamily A member 3 (Tid1), the known transcription factor interactor STAT3, the known extracellular stabilizer CD44, as well as the protein tyrosine phosphatase transmembrane receptor, PTPRF. Several other proteins were also enriched with at least one photo-probe, including VAPA and TMED10 that were found in other interactomics studies, CD166 that was reported to interact and mediate EGFR phosphorylation, as well as several others less known to associate with EGFR in human cells such as PHB, CLPTM1, MX1, STX7 and AP1M1.

### Changes in EGFR Neighborhoods Over 60 Min EGF Treatment

The identified neighborhoods were monitored using ECD ecto-tag MultiMap over 60 min EGF stimulation to evaluate whether the EGFR neighbors remain proximal to EGFR and to identify EGFR neighbors that enter later in the signaling. Significantly fewer proteins were enriched at the 60 min timepoint with all three probes: 76 proteins enriched with aryl-diazirine-biotin, 36 proteins enriched with aryl-azide-biotin, and 24 enriched with phenol-biotin using the same statistical threshold [log 2 (fold enrichment)≥1, P<0.05, at least 2 unique peptides, three biological replicates]. Some proteins were found associated at both short and longer times but to varying degrees along with proteins that were uniquely identified with 5 min or 60 min activation. In the GO analysis, EGFR-associated molecular function terms such as protein binding, enzyme binding and protein-containing complex binding were found among the most enriched together with biological process terms including protein localization, cellular macromolecule localization, vesicle-mediated transport, etc.

The time-dependent enrichment was compared for the top protein hits that were highly enriched in one of the datasets or enriched throughout all datasets via a bubble plot. Rab11a, one of the most enriched proteins using all three photo-probes at 5 min EGF stimulation, showed significantly lower enrichment ratios after 60 min EGF stimulation. This suggests rapid association followed by disassociation and is consistent with the transient role Rab11a has in protein trafficking. Similarly, EPS15 was highly enriched at 5 min using aryl-azide-biotin, but the enrichment ratio significantly dropped at 60 min.

Other proteins showed sustained levels of enrichment upon EGF stimulation such as PTPRF, SRC, ARF6, DNM1, CD44 at both short and long timepoints. Transcription factors such as STAT3, STAT2 and β-catenin (CTNNB) also showed little change in enrichment at 5 min and 60 min. Interestingly, enrichment ratios of several proteins became more prominent at 60 min compared to 5 min. This group of proteins included the protein tyrosine phosphatase non-receptor type 6, PTPN6, also called SHP1, which has been reported to physically interact with EGFR, integrin A2 (ITGA2) and integrin AV (ITAV), cathepsin L (CTSL) which was characterized to mediate EGFR extracellular cleavage and shedding, RHEB which was recently reported to physically interact with EGFR when it enters lysosome, as well as other cell surface proteins TMED10, SCARB1 and BCAM that is not known to associate. It is likely that these proteins are recruited later to EGFR.

To validate these candidate neighbors and confirm that their engagement with EGFR is dependent on ligand activation, a proximity ligation assay was used to visualize the neighbors in situ using PLA. PLA provides spatial proximity information with high sensitivity and is orthogonal to other assays such as co-immunoprecipitation. The PLA was performed on five of the top hits with available and selective antibodies, Rab11a, EPS15, PTPRF, STAT3 and SRC in permeabilized wild-type A549 cells to visualize physical proximity of these proteins with EGFR in the presence of EGF. When comparing ligand-free A549 cells and cells with EGF treatment, significant increases of PLA foci per cell upon EGF activation with Rab11a, EPS15, PTPRF and STAT3 was found. By using flow cytometry as a detection method, the same pattern of EGF-dependent proximity to EGFR with larger cell populations, including EGFR neighbors Rab11a, EPS15, PTPRF, STAT3, SRC were observed, as well as in the additional targets identified in the proteomics datasets, ARF6, DNM1 and CTNNB. While the PLA results cannot provide as high temporal resolution as PLP does, PLA does provide orthogonal support for the physical proximity of these targets with EGFR upon EGF stimulation.

### ICD Ecto-Tag MultiMap Maps Intracellular Neighborhoods

Although the photo-probes used here are in principle cell permeable, it was believed that placing EY on the intracellular domain of the EGFR could trigger greater proximity for identification of intracellular EGFR neighborhoods. A similar PLP workflow where cells expressing EGFR-HaloTag were serum-starved and incubated with EY-HTL was performed. Repeated washout steps were used to remove non-covalently bound EY-HTL so as to reduce non-selective labeling. EGF was added to cells followed by each of the three photo-probes and illuminated with blue light for 2 min to induce biotinylation. Comparable bulk labeling was observed in cells with no ligand, with 5 min EGF activation and 60 min EGF activation by Western blot analysis reflecting effective biotinylation in all states.

The ICD ecto-tag MultiMap workflow was then used to identify the specific proteins enriched with and without EGFR-HaloTag. EGFR was one of the most enriched proteins for all three probes in A549 cells. Also identified were some EGFR neighbor proteins that were found with ECD ecto-tag MultiMap such as GSTP1, SRC, Rab11a, ITA3, ZO1, GSTP1 as well as other new proteins of interest including SHP2 which is a known positive regulator of EGFR signaling, and several LIM domain proteins such as PDLIM5, PDLIM7, LMO7 and LASP1. Proteins enriched upon EGF treatment at 5 min and 60 min timepoints were then compared. A total of 105 proteins enriched with aryl-diazirine-biotin, 59 proteins with aryl-azide-biotin and 229 proteins with phenol-biotin were found at the 5 min EGF timepoint using a standard metrics of log 2 (fold enrichment)≥1, P<0.05, at least 2 unique peptides, and three biological replicates. At the 60 min timepoint, 112 proteins enriched with aryl-diazirine-biotin, 13 proteins with aryl-azide-biotin and 25 proteins with phenol-biotin were found using the same threshold. The ICD ecto-tag MultiMap data had significantly fewer enriched proteins annotated as plasma membrane proteins or cell surface proteins both at 5 min and 60 min compared to those seen from our ECD ecto-tag MultiMap datasets.

Comparative analysis of proteins identified at the 5 min and 60 min timepoints was done for ECD and ICD ecto-tag MultiMap datasets, respectively. The time-dependent enrichment profiles for individual proteins showed similarities. For example, EPS15 was significantly more enriched at the 5 min timepoint compared to the 60 min timepoint while for CD44, ARF6 and STAT3, enrichment levels remained the same. Some proteins with different enrichment profiles between ECD and ICD ecto-tag MultiMap datasets were also found. For example, ICD ecto-tag enrichment data for Tid1 showed more significant changes at 5 min and 60 min than seen in the ECD ecto-tag data. Tid1 is an intracellular mitochondrial-associated protein that reportedly regulates the EGF-stimulated interaction with the HSP70 chaperone system. PTPN6, also called SHP-1, on the other hand, was similarly more enriched at 60 min by ICD ecto-tag using phenol-biotin, possibly for the same reason.

ICD ecto-tag MultiMap additionally provided new EGFR neighbor candidates. This list includes the translocon-associated protein SSRA and Sec61, which are known to form complexes that facilitate the translocation of proteins including EGFR into the nucleus, early endosomal proteins EEA1 and VPS that may engage with EGFR during endocytosis, as well as Rab27A that plays important roles in cellular transport and a less studied protein TM9S2.

The enriched hits seen in ICD ecto-tag MultiMap were further validated using Western blots. Western blots depend on the quality of the antibodies available and may not have the sensitivity of MS. Nonetheless, the increase in biotinylation of Rab11a, EPS15, SRC, PTPN6 and DNM1 with aryl-diazirine-biotin enrichment, as well as PTPRF, STAT3 with aryl-azide-biotin enrichment, and ARF6, ITGA2, CD44 with phenol-biotin enrichment at the 5 min timepoints was confirmed, providing orthogonal support for direct association of neighboring proteins with EGFR. A monotonic trend of enrichment levels from 5 min to 10 min, 30 min and 60 min EGF activation was also found. The combination of photo-proximity labeling proteomics and Western blot analysis provide mutually supportive evidence that the identified EGFR neighbors are in physical proximity.

The neighborhoods identified by ECD and ICD ecto-tagged MultiMap were compared. They differ substantially with less than 10% overlap of proteins identified. It is believed this reflects triggering of the photo-proximity labeling from outside versus inside and highlights the complementary nature of these two methods. The collected datasets were also compared with published EGFR and STS1 interactome datasets that used AP-MS pull-downs and APEX2 in cells other than A549 cells. While there were some common proteins, there was significant differences likely due to different cell lines, treatment conditions, different experimental workflows and instruments used.

EGFR Neighbors are Associated with EGF-Activated Functions

EGFR is known to undergo staged cytosolic signaling events over an hour or so after EGF ligand activation, including phosphorylation, internalization, degradation and transcriptional regulation. To determine if targets identified by ECD and ICD ecto-tag MultiMap were regulating these processes knock down strategies were designed against the most prominent candidate EGFR neighbors from the datasets via CRISPR-mediated and siRNA-based genetic perturbation, and individually knocked down seven genes of candidate interactors including SRC, ARF6, PTPRF, EPS15, RAB11A and DNM1 as well as EGFR in A549 cells. Western blot analysis confirmed selective knockdown of each target to over 50%, with the exception of SRC; the SRC gene is known to be essential and thus was limited to 30% knockdown. The gene knockdowns did not alter the overall levels of EGFR expression, suggesting they did not perturb EGFR expression or trafficking.

Phosphorylation at tyrosine sites, including Tyr1086, Tyr845, Tyr1045, Tyr978, Tyr1068 and Tyr992, is known to be an immediate and critical regulatory mechanism behind EGFR signaling cascades. The differential kinetic patterns of phosphorylation at the different sites in WT A549 cells was tested. As expected, a significant increase of phosphorylation at all sites at 5 min EGF stimulation followed by dephosphorylation responses at Tyr845, Tyr1045, and Tyr992 and sustained phosphorylation at Tyr1086 and Tyr1068 sites at by 60 min was observed. Knockdowns of SRC or ARF6 significantly lowered levels of EGFR phosphorylation at Tyr1086 and Tyr1068, which are the major autophosphorylation sites involved in the MAPK signaling cascade. Additionally, phosphorylation at Tyr978 site decreased with SRC and ARF6 knockdowns. SRC knockdown also had a mild influence on phosphorylation at Tyr845 and Tyr1045 sites. Interestingly, knockdown of the gene for the phosphatase PTPRF, a known tumor suppressor, led to increased early phosphorylation at Tyr845 and Tyr1068 and late phosphorylation of Tyr1086 and Tyr992 while Tyr1045 and Tyr978 were largely unaffected.

Time-dependent EGFR internalization was tracked by monitoring cell-surface EGFR by flow cytometry at different timepoints of EGF activation at 37° C. With wild-type A549 cells, EGFR was internalized rapidly starting from 5 min EGF activation and was kept internalized at the 60 min and 3 hour timepoints. After three hours of EGF stimulation, >60% EGFR internalization in wild-type A549 cells was observed; knockdown of RAB11A, EPS15, DNM1 or ARF6, significantly reduced internalization of EGFR to only ˜20%. These data suggest that Rab11a, EPS15, DNM1 and ARF6 contribute to EGFR internalization upon EGF activation.

After internalization, EGFR can be degraded by 16 hr which is known to regulate EGFR activity. Knockdowns of EPS15 and DNM1 caused significant reduction in EGFR degradation compared to WT cells in the presence of cycloheximide, suggesting they may be involved in lysosomal trafficking. Interestingly, knockdowns of RAB11A and ARF6 had no significant effect at the 1 hour and 3 hour timepoints but slightly enhanced EGFR degradation at the 16 hour timepoint. This may be contributed by Rab11a's ability to mediate EGFR recycling from endosome back to membrane. ARF6 may additionally regulate EGFR level by engaging the EGFR sorting process from Golgi to membrane. These data support the roles of these proteins in promoting EGFR trafficking that ultimately leads to EGFR turnover and homeostasis.

Activated EGFR also drives transcription through the regulation of a number of transcription factors including STAT3 and β-catenin (CTNNB). As expected, both STAT3 and β-catenin were enriched through the ECD and ICD ecto-tag MultiMap upon EGF stimulation, indicating that they are in physical proximity. To further confirm the regulatory function of EGFR in the A549 cells, EGFR knockdown was conducted and STAT3 and β-catenin transcriptional activities via qPCR analysis was monitored. When WT-A549 cells were stimulated with EGF, increased transcriptional activities of STAT3 and β-catenin was observed, leading to increased levels of the β-catenin-specific target gene AXIN2, the STAT3-specific target gene ZEB1, and the shared β-catenin/STAT3 target gene cMYC. On the other hand, EGFR inhibition with cetuximab reduced the gene expression for both STAT3 and β-catenin systems. When EGFR is knocked down, RNA levels of all these genes were significantly reduced and minimal induction was observed in the presence of EGF that phenocopied the pattern with EGFR inhibition. These data support the hypothesis that transcriptional activities of both STAT3 and β-catenin are simultaneously modulated by EGFR. Collectively, these functional validation studies show that knockdown of candidate neighbors can affect EGFR signaling phenotypes including phosphorylation, internalization, degradation and transcriptional regulation.

What is claimed is:

1. A method of labeling a protein with a photoreactive probe comprising:
admixing a protein, a photoreactive probe, and photocatalyst to form a mixture; and
irradiating the mixture with light having a wavelength in a range of about 410 nm to about 570 nm;
wherein the photocatalyst has a structure according to formula (I): 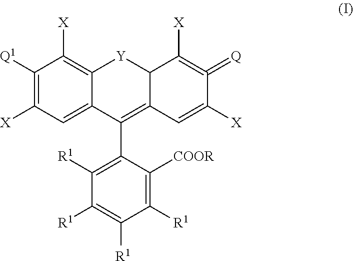
wherein:
each X is independently selected from H, Br, I, F, and Cl;
Q is O or NR^{a};
Q¹ is OH, OR^{a}, NHR^{a}, or NR^{a}₂;
each R¹ is independently selected from H, Cl, F, I, and Br;
Y is O, S, or Si(R^{a})₂;
R is H or a cation; and
each R^{a} is independently selected from C₁-C₁₂ alkyl; and
wherein the photoreactive probe comprises a photoreactive group coupled to a second moiety.

2. The method of claim 1, wherein the photoreactive group is selected from the group of a diazirine, an aryl-azide, a phenol, a hydrazide, and a combination thereof.

3. The method of claim 1 or claim 2, wherein the second moiety comprises biotin, a fluorophore, a crosslinking reagent, or a bioorthogonal handle such as an azide, an alkyne, a tetrazine, a dibenzocyclooctyne (DBCO), or a trans-cyclooctyne (TCO).

4. The method of claim 1 or claim 2, wherein the second moiety comprises an enzyme, a peptide, a protein, or a bioactive molecule.

5. The method of claim 1 or claim 2, wherein the photoreactive probe is selected from the group of:

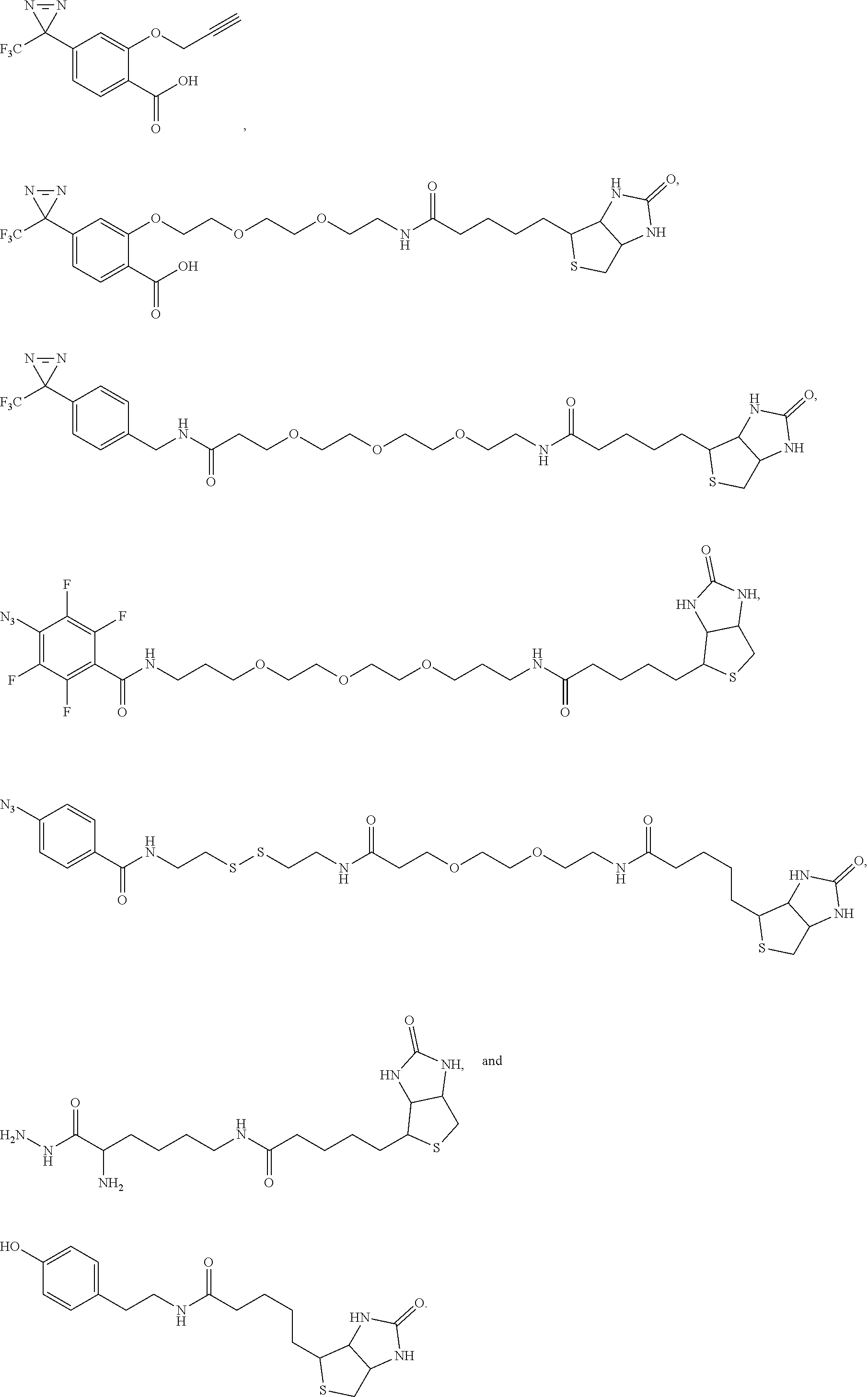

6. The method of any one of the preceding claims, wherein the mixture includes two or more photoreactive probes.

7. The method of claim 6, wherein the two or more photoreactive probes are different from each other in that the photoreactive probes have different photoreactive groups.

8. The method of any one of the preceding claims, wherein the light has a wavelength in a range of about 410 nm to 470 nm.

9. The method of any one of the preceding claims, wherein the light is a green light having a wavelength in a range of about 500 nm to about 570 nm.

10. The method of any one of the preceding claims, wherein R is a cation.

11. The method of any one of the preceding claims, wherein at least one X is H, F, Br, or I.

12. The method of any one of the preceding claims, wherein all X are H, Br or I.

13. The method of any one of the preceding claims, wherein all X are Br.

14. The method of any one of claims 1 to 12, wherein all X are I.

15. The method of any one of claims 1 to 12, wherein all X are H.

16. The method of any one of claims 1 to 12, wherein at least two X are F.

17. The method of any one of the preceding claims, wherein Y is O.

18. The method of any one of claims 1 to 16, wherein Y is S.

19. The method of any one of claims 1 to 16, wherein Y is Si(CH₃)₂.

20. The method of any one of the preceding claims, wherein all R¹ are H.

21. The method of any one of claims 1 to 19, wherein all R¹ are Cl.

22. The method of any one of claims 1 to 19, wherein all R¹ are F.

23. The method of any one of claims 1 to 9, wherein the photocatalyst is selected from the group of: 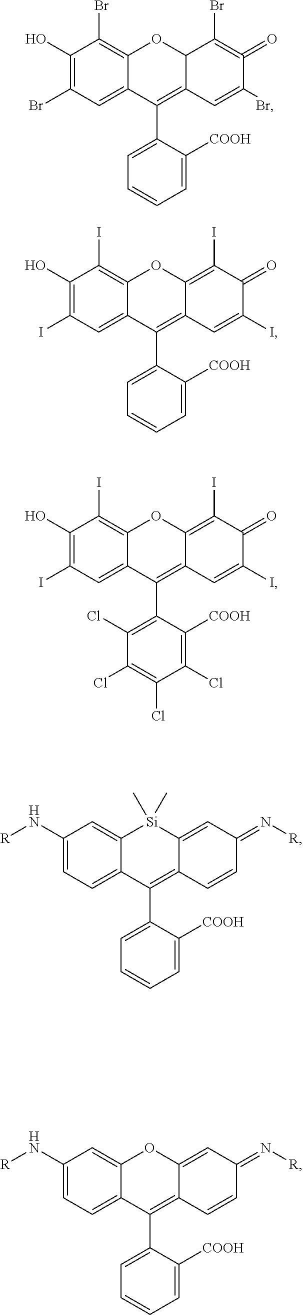 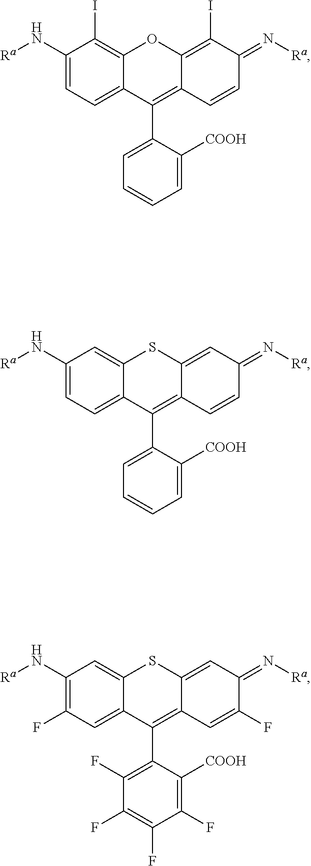
and salts thereof.

24. The method of any one of the preceding claims, wherein the admixing comprises combining the protein, the photoreactive probe, and the photocatalyst in solution.

25. The method of claim 24, wherein the solution is an aqueous solution.

26. The method of claim 25, wherein the aqueous solution comprises a buffer.

27. The method of any one of the preceding claims, wherein the admixing takes place at a temperature in a range of about 4° C. to about 60° C., or about 4° C. to about 50° C., or about 4° C. to about 37° C., or about 20° C. to about 25° C., or about 4° C., about 20-25° C., or about 37° C.

28. A method of proximity labeling proteins on a cell surface comprising:
admixing
a cell having a surface membrane, the surface membrane having a transmembrane target protein and the cell having one or more further proteins, wherein the transmembrane target protein is coupled to an antibody-photocatalyst conjugate; and
a photoreactive probe to form a mixture; and
irradiating the mixture with light having a wavelength in a range of about 410 nm to about 570 nm to thereby label at least a portion of the one or more further proteins with the photoreactive probe and provide labeled proteins;
wherein the photocatalyst has a structure according to formula (I): 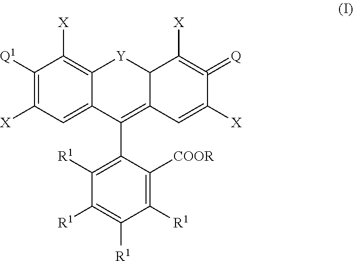
wherein:
each X is independently selected from H, Br, I, F, and Cl;
Q is O or NR^{a};
Q¹ is OH, OR^{a}, NHR^{a}, or NR^{a}₂;
each R¹ is independently selected from H, Cl, F, I, and Br;
Y is O, S, or Si;
R is H or a cation; and
each R^{a} is independently selected from C₁-C₁₂ alkyl; and
wherein the photoreactive probe comprises a photoreactive group coupled to a second moiety.

29. The method of claim 28, wherein the one or more further proteins are in proximity of the transmembrane target protein.

30. The method of claim 29, wherein the one or more further proteins are within energy transfer range of the transmembrane target protein.

31. The method of claim 29, wherein the one or more further proteins are within about 100 Å to about 3000 Å from the transmembrane target protein.

32. The method of any one of claims 28 to 31, wherein the transmembrane target protein is directly coupled to the antibody of the antibody-photocatalyst conjugate.

33. The method of any one of claims 28 to 31, wherein the transmembrane target protein is coupled to the antibody-photocatalyst conjugate through an ecto-tag, wherein the ecto-tag is directly coupled to the transmembrane target protein and the antibody of the antibody-photocatalyst conjugate is directly coupled to the ecto-tag.

34. The method of claim 33, wherein the ecto-tag is selected from the group of a FLAG tag, a myc tag, a halo tag, a spy tag, an ALFA tag, a His tag, GFP tag, EPEA tag, or an HA tag.

35. The method of any one of claims 28 to 34, wherein the transmembrane target protein comprises epidermal growth factor receptor (EGFR), CUB domain-containing protein 1 (CDCP1), B-lymphocyte antigen CD19 (CD19), human epidermal growth factor receptor 2 (HER2), immunoglobin E (IgE), or B-cell maturation antigen (BCMA).

36. The method of any one of claims 28 to 36, wherein the antibody comprises cetuximab and the transmembrane target protein comprises an epidermal growth factor receptor.

37. The method of any one of claims 28 to 35, wherein the antibody comprises trastuzumab and the transmembrane target protein comprises HER2.

38. The method of any one of claims 28-37, wherein the photoreactive group is selected from the group of a diazirine, an aryl-azide, a phenol, a hydrazide, and a combination thereof.

39. The method of any one of claims 28-38, wherein the second moiety comprises biotin, a fluorophore, a crosslinking reagent, or a bioorthogonal handle such as an azide, an alkyne, a tetrazine, a dibenzocyclooctyne (DBCO), or a trans-cyclooctyne (TCO).

40. The method of any one of claims 28-38, wherein the second moiety comprises an enzyme, a peptide, a protein, or a bioactive molecule.

41. The method of any one of claims 28-38, wherein the photoreactive probe is selected from the group of:

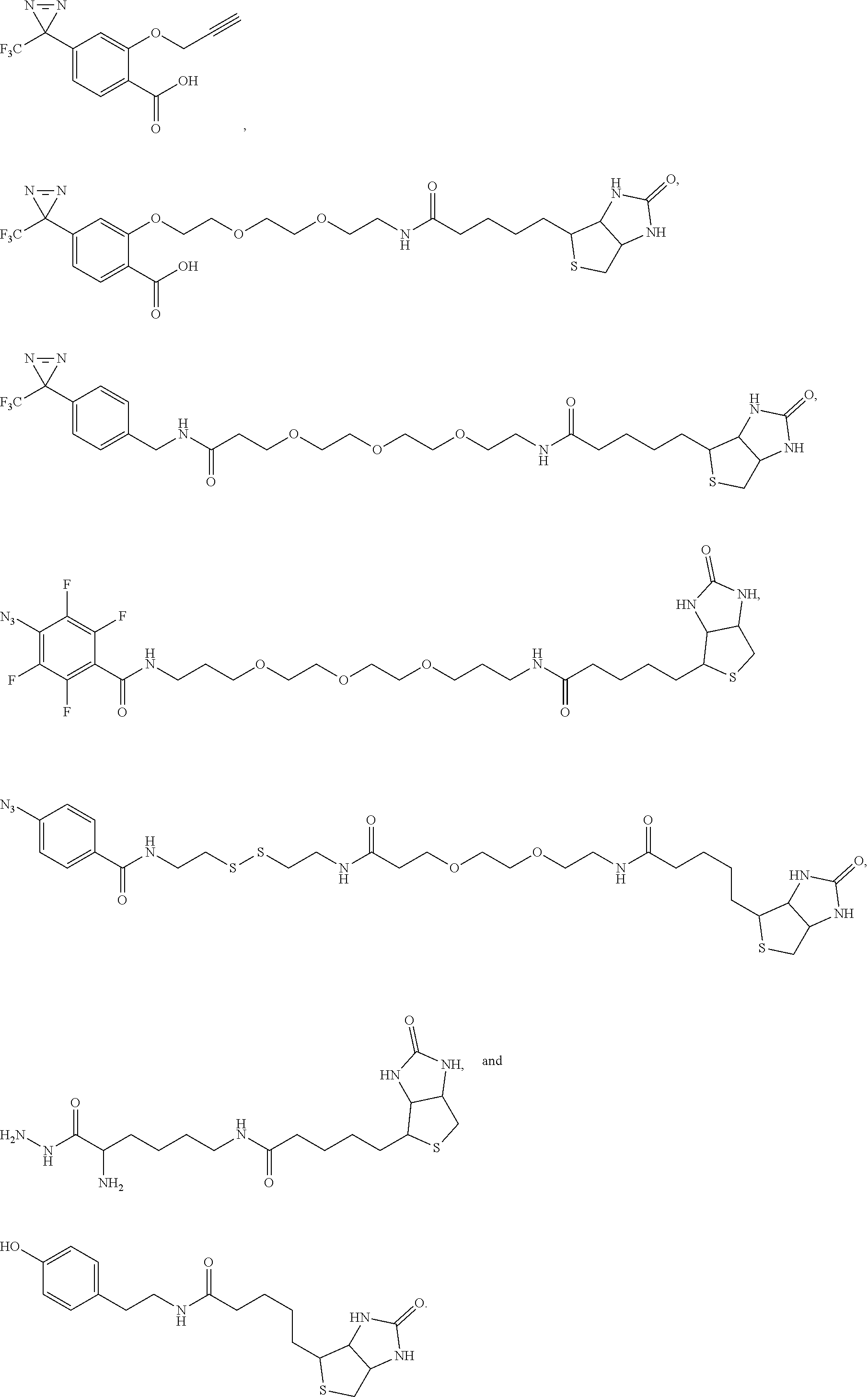

42. The method of any one of claims 28-41, wherein the mixture includes two or more photoreactive probes.

43. The method of claim 42, wherein the two or more photoreactive probes are different from each other in that the photoreactive probes have different photoreactive groups.

44. The method of any one of claims 28-43, wherein the light has a wavelength in a range of about 410 nm to 470 nm.

45. The method of any one of claims 28 to 43, wherein the light is a green light having a wavelength in a range of about 500 nm to about 570 nm.

46. The method of any one of claims 28-45, wherein R is a cation.

47. The method of any one of claims 28-46, wherein at least one X is H, F, Br or I.

48. The method of any one of claims 28-47, wherein all X are H, Br or I.

49. The method of any one of claims 28-48, wherein all X are Br.

50. The method of any one of claims 28-48, wherein all X are I.

51. The method of any one of claims 28 to 48, wherein all X are H.

52. The method of any one of claims 28-47, wherein at least two X are F.

53. The method of any one of claims 28-52, wherein Y is O.

54. The method of any one of claims 28-52, wherein Y is O.

55. The method of any one of claims 28-52, wherein Y is Si(CH₃)₂.

56. The method of any one of claims 28 to 55, wherein all R¹ are H.

57. The method of any one of claims 28 to 55, wherein all R¹ are Cl.

58. The method of any one of claims 28 to 55, wherein all R¹ are F.

59. The method of any one of claims 28 to 45, wherein the photocatalyst is selected from the group of: 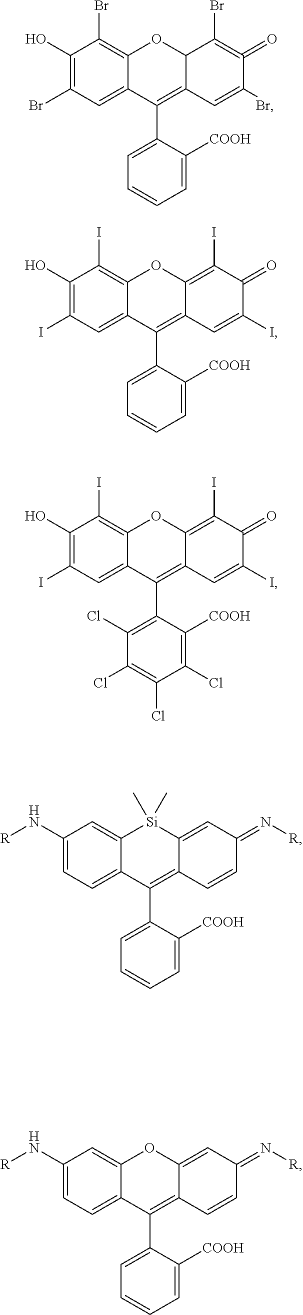 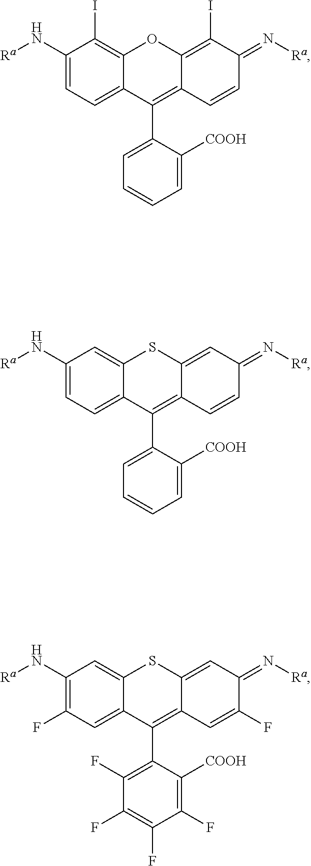
and salts thereof.

60. The method of any one of claims 28-59, further comprising characterizing the labeled proteins.

61. The method of claim 60, wherein characterizing the labeled proteins comprises mass spectrometry, fluorescence imaging, DNA barcoding, or a combination thereof.

62. The method of any one of claims 28 to 61, wherein the admixing comprises combining the cell and the photoreactive probe in solution.

63. The method of claim 62, wherein the solution is an aqueous solution.

64. The method of claim 63, wherein the aqueous solution comprises a buffer.

65. The method of any one of claims 28 to 64, wherein the admixing takes place at a temperature in a range of about 4° C. to about 60° C., or about 4° C. to about 50° C., or about 4° C. to about 37° C., or about 20° C. to about 25° C., or about 4° C., about 20-25° C., or about 37° C.
